(19) United States
(12) Patent Application Publication
(10) Pub. No.: US 2025/0257344 A1
(43) Pub. Date: Aug. 14, 2025

(54) METHOD OF REGULATING ALTERNATIVE POLYADENYLATION IN RNA

(71) Applicant: THE REGENTS OF THE UNIVERSITY OF CALIFORNIA, Oakland, CA (US)

(72) Inventors: Eugene Yeo, San Diego, CA (US); Pratibha Jagannatha, Fremont, CA (US)

(21) Appl. No.: 18/833,339

(22) PCT Filed: Jan. 31, 2023
(86) PCT No.: PCT/US2023/012047

§ 371 Date: Jul. 25, 2024

Related U.S. Application Data
(60) Provisional application No. 63/305,462, filed on Feb. 1, 2022.

(51) Int. Cl. C12N 9/22; C12N 15/11; C12N 15/113

(52) U.S. Cl. CPC C12N 9/226; C12N 15/11; C12N 15/113; C12N 2310/11; C12N 2310/20

(57) ABSTRACT

Provided herein are methods of regulating alternative polyadenylation (APA) of a target RNA in a cell, increasing stability of a target RNA in a cell, preventing degradation of a target RNA in a cell, preventing degradation of a target RNA in a cell, modifying localization of a target RNA in a cell, or increasing synthesis of a protein encoded by a target RNA in a cell, by administering to the cell an RNA regulation unit, wherein the RNA regulation unit comprises an RNA binding protein (RBP) and a gene-targeting agent, wherein the RNA binding protein binds proximal to a poly(A) signal and/or site.

### CROSS-REFERENCE TO RELATED APPLICATION

This application claims priority under 35 U.S.C. § 119(e) and under the Paris Convention to U.S. Provisional Patent Application No. 63/305,462, filed on Feb. 1, 2022. The disclosure of U.S. Provisional Patent Application No. 63/305,462, is considered part of the disclosure of this application, and is incorporated herein by reference in its entirety.

### BACKGROUND

Alternative polyadenylation (APA) is a post-transcriptional processing mechanism important in gene regulation. It involves the differential usage of poly(A) sites in a gene followed by cleavage and polyadenylation. This mechanism is widespread with an estimated 70% of human genes shown to contain multiple poly(A) sites. Most importantly, it has the potential to regulate the fate of RNA stability, translation, and localization through the differential usage of poly (A) sites.

One way in which APA site selection is regulated is through RNA binding proteins (RBPs) which can bind to RNA targets, influence poly (A) site usage, and in turn, impact RNA stability, translation, and localization. In addition to RBPs that participate in the core cleavage and polyadenylation complex, studies highlight select examples of RBPs and their ability to modulate APA site usage alluding to their potential to be used to regulate APA site usage in the form of a molecular tool or RNA therapeutic. These studies have generally employed informative but low throughput methods that couple the knockdown of a specific RBP with an experimental method to observe the effects on poly (A) site section. Therefore, there has not yet been a comprehensive list of RBPs with known binding-locale specific effects on poly(A) site selection available.

### SUMMARY

Provided here are methods of regulating alternative polyadenylation (APA) of a target RNA in a cell, the methods comprising, or consisting essentially of, or yet further consisting of, assembling an RNA regulation unit, wherein the RNA regulation unit comprises an RNA binding protein (RBP) and a gene-targeting agent, wherein the RNA binding protein binds proximal to a poly(A) signal and/or site; delivering the RNA regulation unit into the cell, wherein the RNA regulation unit regulates alternative polyadenylation (APA) of the target RNA in the cell; and detecting a change in the target RNA translation.

Provided herein are methods of increasing stability of a target RNA in a cell, the methods comprising, or consisting essentially of, or yet further consisting of, assembling an RNA regulation unit, wherein the RNA regulation unit comprises an RNA binding protein (RBP) and a gene-targeting agent, wherein the RNA binding protein binds proximal to a poly(A) signal and/or site, wherein the RNA regulation unit increases stability of the target RNA in the cell; delivering the RNA regulation unit into the cell; and detecting a change in the target RNA translation.

Provided herein are methods of preventing degradation of a target RNA in a cell, the methods comprising, or consisting essentially of, or yet further consisting of, assembling an RNA regulation unit, wherein the RNA regulation unit comprises an RNA binding protein (RBP) and a gene-targeting agent, wherein the RNA binding protein binds proximal to a poly(A) signal and/or site, wherein the RNA regulation unit prevents degradation of the target RNA in the cell; delivering the RNA regulation unit into the cell; and detecting a change in the target RNA translation.

Provided herein are methods of modifying localization of a target RNA in a cell, the methods comprising, or consisting essentially of, or yet further consisting of, assembling an RNA regulation unit, wherein the RNA regulation unit comprises an RNA binding protein (RBP) and a gene-targeting agent, wherein the RNA binding protein binds proximal to a poly(A) signal and/or site; delivering the RNA regulation unit into the cell; and detecting change in the target RNA translation, wherein the RNA regulation unit modifies localization of the target RNA in the cell.

Provided herein are methods of increasing synthesis of a protein encoded by a target RNA in a cell, the methods comprising, or consisting essentially of, or yet further consisting of, assembling an RNA regulation unit, wherein the RNA regulation unit comprises an RNA binding protein (RBP), and a gene-targeting agent, wherein the RNA binding protein binds proximal to a poly(A) signal and/or site, wherein the RNA regulation unit increases synthesis of the protein encoded by the target RNA in the cell; delivering the RNA regulation unit into the cell; and detecting a change in the target RNA translation.

Provided herein are RNA regulation units, wherein an RNA regulation unit comprises, or consisting essentially of, or yet further consisting of, an RNA binding protein (RBP), and a gene-targeting agent, wherein the RNA binding protein binds proximal to a poly(A) signal and/or site of a target RNA.

In some embodiments, the RBP binds to a plurality of poly(A) signals and/or sites. In some embodiments, the RNA binding protein binds to the poly(A) site. In some embodiments, the gene-targeting agent comprises CRISPR components. In some embodiments, the gene-targeting agent comprises a Cas RNA targeting system. In some embodiments, the Cas RNA targeting system comprises inactive Cas13 (dCas13). In some embodiments, the Cas RNA targeting system comprises inactive RNA-targeting Cas9 (dCas9). In some embodiments, the gene-targeting agent comprises a non-Cas RNA-targeting system. In some embodiments, the non-Cas RNA-targeting system comprises a CRISPR-cas inspired RNA targeting system (CIRTS). In some embodiments, the gene-targeting agent comprises small molecules, or engineered protein domains. In some embodiments, the engineered protein domain comprises a PUF domain. In some embodiments, the gene-targeting agent comprises shRNAs, siRNAs, antisense oligonucleotides (ASOs), or microRNA mimics. In some embodiments, the delivering step (b) comprises lipofection. In some embodiments, the delivering step (b) comprises a virus-based delivery. In some embodiments, the virus-based delivery comprises adeno-associated virus or lentivirus. In some embodiments, the target RNA is an endogenous mRNA. In some embodiments, the target RNA is a non-coding RNA. In some embodiments, the RBP binds upstream of a poly(A) signal and/or site of the target RNA, and wherein the RBP activates poly(A) site selection of the target RNA. In some embodiments, the RBP is CPSF5, RNPS1, CPSF6, CSTF1, RBM11, TRNAU1AP, RBM14, MBNL1, PRRC2B, EIF4B, LGALS1, LUC7L, APOBEC3A, FUBP1, CDC40, UBE2I, SRP68, NGRN, ZRANB2, GRB2, RBM5, ZC3H18, PRPF40A, TIAL1, RBM10, ZC3HAV1, RPS10, YTHDF1, EIF4A3, IGF2BP3, SAMD4A, PNN, CLK2, PRPF4, RPS28, EIF4H, RY1, LARP4B, EIF3G, FLYWCH2, CIR1, WDR6, SMNDC1, SLBP, GTSF1, U2AF2, PRPF4, RBBP6, SRSF8, MBNL2, SRSF9, PCBP1, SBDS, PPIA, RPS19BP1, ISY1, CPSF6, DNAJC17, TOB2, RBM38, RPS21, LGALS3, CPSF7, DDX6, TRA2A, BUD13, SNRPA, STXBP1, RPL30, LSM1, GNB2L1, SARNP, HFM1, CNOT3, METTL16, LSM5, CCDC75, CHAF1B, STAU1, AHNAK, RBM8A, MAZ, NOSIP, PARN, SPATS2L, SNRNP40, YWHAG, LSM10, SNRNP70, PHF5A, RBFOX1, TPD52L2, RBM18, BCDIN3D, U2AF1, CSTF2T, SFPQ, SUPT5H, LARP4, EDC3, ASCC1, MKI67IP, EIF4A1, PDIA3, SSRP1, HNRNPCL1, PAPOLG, EDF1, RBM12B, or DCD. In some embodiments, the RBP binds upstream of a poly(A) signal and/or site of the target RNA, and wherein the RBP inhibits poly(A) site selection of the target RNA. In some embodiments, the RBP is RBPMS, RPL23, DHX16, NDUFV3, LUC7L3, RPL31, SRSF3, RPP21, SUMO1, FKBP4, FASN, DDX39A, EZR, RBFOX2, ZCRB1, RAN, AHNAK, SRSF11, SERBP1, PIWIL4, FUBP3, ERCC3, EIF2C2, DNAJC2, RBM26, NUDT16L1, SLIRP, APOBEC3C, LSMD1, SRBD1, RPS3A, SSBP1, WDR3, RPL36, SUGP1, STIP1, SLC3A2, IFIT2, RPL23, ELMOD3, CPSF1, APOBEC3G, GLRX3, XPO5, COA6, CPSF4, HSPD1, PTRF, DCP2, XPO5, RPS15A, DDX20, GRN, PHF6, SUGP2, RBMS1, SF1, RBM33, SRPR, NAA15, DCN, DIS3, SF3B2, TRAP1, MTPAP, FTO, C1orf131, RPL3, IFI16, USP36, RBM25, EXOSC3, NPM1, MRPS5, CCDC9, SEC61B, ZCCHC7, FAM120A, DNTTIP2, NOLC1, EIF2S2, SAFB2, SNRPE, ILF2, RDBP, THOC5, DDX1, MRPS11, PUS7L, RPL6, NPM1, SF3B4, CLP1, TRIM56, CALR, DHX58, RPS6, ARL6IP4, PRPF38B, APOBEC3B, REPIN1, RBM45, MEPCE, CNOT6, EIF2C2, DMGDH, NOA1, ASS1, RPS3, EIF5B, RBM17, EXOSC10, NSA2, DCP1A, SART3, APEH, TEFM, BTG4, BCCIP, PNLDC1, EIF3H, HSP90AA1, TSR1, CLASRP, CPNE3, PSMD4, FAM98A, PUF60, G3BP1, PRPF19, RPS6, RBFOX2, CPEB1, SEC63, RPL15, NHP2, ZCCHC11, HSPA9, RTN4, NONO, ZCCHC7, EIF2AK2, RPL21, DHX40, ELAVL4, TUFM, RPL5, DHX8, SRFBP1, RPS20, SNRPG, PWP2, THOC3, SUCLG1, PDIA4, ZMAT3, ZYX, DHX36, FAM98A, DDX19B, ANXA2, POLK, FTSJ3, LARP1B, ANXA2, C1D, ACAA2, PSMC1, DDX18, PPP1CB, PSPC1, LARP4, CNOT10, FANCM, MRPL11, DDX27, DDX11, SEC63, HEATR1, NOP2, RPS7, DYNC1LI1, SAMSN1, DHX35, EIF3L, PABPN1L, RRS1, HADHB, NUDT16, SLC7A9, CPSF4, MRPL37, ERAL1, RPL8, RNMTL1, SRP14, PPAPDC2, NPM3, CORO1A, STAU2, RPL28, CELF4, RPUSD4, YTHDC1, DIMT1, HADHB, TBRG4, DDX55, FASTK, ALDH6A1, CPSF2, SF3B3, SNRPB2, PUS7, RPL35, DDX53, MDH2, FXR2, ILF3, METTL3, TRMT2A, HSPA8, SF3A1, TPT1, FDPS, CLK3, RRP8, RPS8, GPANK1, LARP6, TDRD9, TARDBP, ATP5C1, MRPS11, PCBP4, RBM26, UTP11L, RPL4, CCDC137, PINX1, RNMTL1, FZD8, NAP1L3, POLR2G, DDX31, RPS2, DDX41, SRPR, RBM28, RPN1, RPS24, NXF1, G3BP2, U2AF1, RTCA, RBM46, BYSL, DYNC1H1, PRR3, POLR3E, RBM6, PUF60, RTN4, GANAB, TSR1, TRUB2, DDX28, TERT, DIEXF, NOP58, APOBEC3D, IFIH1, QKI, KIAA1324, EEF1A1, RALY, CNOT7, RPS4X, DDX39A, FASTKD2, NOL10, DDX11, PES1, EFTUD2, PPIL4, RSL1D1, SNRPN, KRR1, MRPL32, ELAVL2, NANOS3, CIRBP, CCDC59, DDX49, ZFC3H1, RPL15, ALDH18A1, HERC5, MRM1, CNBP, RPLP0, KRT18, TOP3B, FSCN1, SLC25A5, SURF6, CPSF4L, RPL14, MRPL39, THOC6, PKM, RPL3, NOL7, DDX43, GSPT2, NOP16, NOL12, RBM12, ABT1, KRR1, HTATSF1, KIAA0020, NOP16, RPL27, UTP23, BST2, RBM47, EIF2C1, MAK16, PTCD1, DDX60, GPATCH4, PA2G4, DDX55, LARP7, GTF2E2, USP32, THUMPD1, MRPL30, DDX59, MRPS24, UCHL5, NXF3, AIMP1, THOC4, GLTSCR2, DHX33, KHDRBS2, BMS1, DDX1, NOC2L, MAP4, SAMD4A, YARS, RBM34, HDGF, PIWIL1, TRMU, ZFP36L1, TROVE2, ACTN4, RBM19, AK8, XRCC6, FAM208A, SRSF10, NAT10, SNRNP35, RPL7, MRPL3, NOP9, ARL6IP4, DDX52, HNRNPD, MRPS23, RPL15, HSP90AB1, RBM4, DHX30, HNRNPA1, NXF2, THRAP3, ADAD2, RPL13A, RPL35, HMGB2, HIST1H1C, EIF2C3, HNRNPC, ILF3, RBM15B, RPL19, MRPL4, DUS2L, TRIP6, GTPBP4, EEF2, DDX54, RPL23A, RRP36, PPAN, PATL1, REXO4, DND1, NOP56, RPS11, RBM15, RPS4X, MRPS15, HNRNPR, PIWIL2, SYNCRIP, SNRPB, RPL32, or FBL. In some embodiments, the RBP binds downstream of a poly(A) signal and/or site of the target RNA, and wherein the RBP activates alternative polyadenylation of the target RNA. In some embodiments, the RBP is CSTF1, TOB2, PRRC2B, PARN, RBM11, DDX17, GTSF1, DDX5, RBM10, SNRPA, RBM22, CNOT4, RBM14, MBNL1, SUPT5H, ZC3HAV1, RBM4, CNOT2, SAMD4A, CDC40, HNRNPH1, SRP68, EIF4B, CSTF2T, LGALS3, NANOS3, SFPQ, CIR1, PPP1CA, ZMAT3, PRPF4, PRPF4, SCAF8, TRNAU1AP, EDC3, PCBP3, LSM1, PPIA, TPD52L2, RBM5, APOBEC3A, LGALS1, CDC42EP4, DZIP3, HNRNPH2, STAU2, PCBP1, GRB2, NUP35, EIF4H, BTG1, WDR6, THUMPD1, RSRC1, SMNDC1, SYMPK, NONO, MBNL2, MAZ, STAU1, HNRNPF, TFIP11, SMN1, AHNAK, YTHDF1, NGRN, METTL16, SNRPG, SF3A2, STAU2, YWHAE, YWHAG, SFRS2IP, PEG10, HRSP12, EXOSC10, CDC42EP4, LSM7, ZC3H7A, SNRPN, RBM42, AHNAK, GSPT1, SMN1, MECP2, SRSF9, QARS, ASCC3, CUGBP1, ZFC3H1, NMD3, SNRPB, SRSF2, NUSAP1, RBM38, CORO1A, SFRS17A, BTG4, GTPBP10, SNW1, or CPSF6. In some embodiments, the RBP binds downstream of a poly(A) signal and/or site of the target RNA, and wherein the RBP inhibits poly(A) site selection of the target RNA. In some embodiments, the RBP is FZD3, PPIG, CSTF3, WDR36, DHX8, SLIRP, CNOT10, SOX21, MRPS11, PURG, ADK, TRAP1, LSM4, NGDN, DYNC1H1, FLYWCH2, NPM1, IGF2BP1, ASS1, GNL2, RBPMS, LSM10, FAM46A, TPT1, RNMTL1, LARP4, CPSF5, LUC7L3, NAA15, RBM3, TPT1, ELAC2, RPGR, PNO1, UTP3, SNRPB2, HIST1H1C, ASCC1, SART3, EIF2C3, EIF4A1, EIF3L, MRPS15, LUC7L, SNRPE, LIN28A, CNOT10, NHP2, PARP12, HADHB, MRPS30, XPO5, RPS15A, NPM1, PURB, MTPAP, MRPL11, COA6, GLE1, NUDT16, GRN, RBM17, DHX57, PHF5A, SMG6, SARNP, SUGP2, TSR1, KRR1, C1D, ESRP1, PSMD4, SRSF10, TRMT10C, ZGPAT, BOLL, SPATS2L, MRPS24, RBM26, RPL6, CLASRP, DDX59, SEC63, EIF2S2, RPL23, DHX37, CPEB1, SNRPF, MRPS31, PNLDC1, MRPS23, RPS3A, TEFM, CCT4, POLR2G, RPS7, CNBP, FUBP1, SRPR, DDX60, NAP1L3, RPL22, EIF3C, DDX27, NOA1, LARP1B, FDPS, USP36, CMSS1, CCNL2, RPP30, SF3B3, MEPCE, SBDS, RPUSD4, U2AF2, RPL21, THOC3, FXR2, RPL6, LSM6, FAM98A, WDR33, DUS2L, THOC5, TRIM39, FAM208A, RRP7A, MRPS35, P4HB, LARP1, MAK16, RBM4, EIF2C2, ARL6IP4, FZD8, FTSJ3, PUS7, IFIT2, EIF2C2, S100A4, DHX58, TRIM56, SRPR, SERBP1, MRPL30, GTF2F1, LSMD1, TFB1M, FKBP3, PHF6, METTL3, ALDH18A1, ZFC3H1, GSPT2, GTPBP4, EXOSC3, PRPF6, RC3H2, NSUN2, DHX35, DCN, ANXA2, DHX40, SAFB2, RPS6, IFIH1, DDX43, NOL12, GFM1, RPS20, PUF60, PIWIL1, RBM8A, LARP6, HSP90AB1, MRPS5, MAP4, MRPL37, PRPF19, XRN2, HFM1, ALDH6A1, FTO, SPATS2, TRMU, USP32, TROVE2, RPS15A, XRCC6, DNAJC17, NOP58, RPL7, RPL10A, DDX1, C16orf88, APOBEC3B, PPAPDC2, HDGF, C1orf131, SUPV3L1, RPS3, TDRD9, RALYL, CPSF2, SUCLG1, DDX20, RPN1, RPP25, PTRH1, EEF1A1, FAM32A, SEC63, CNOT6, ILF3, SRFBP1, TRIP6, NOSIP, FSCN1, NOB1, RPS6, GNL2, SRPK2, EIF3D, RPL15, TRMT6, RPL8, NOP2, CHD2, CIRBP, RBMS1, SERBP1, NOP16, FASTKD2, CPSF4, DDX11, CHTOP, PWP2, SREK1, LSM3, BCCIP, BYSL, SECISBP2, TRMT2A, REPIN1, CIRH1A, MRTO4, RPL19, HADHB, NOL10, SYF2, MFAP1, APOBEC3C, PRPF38B, RPS2, NSA2, CPSF3L, SNRNP35, THOC6, DDX11, RPL13A, DDX31, NPM1, BST2, PRPF40A, PES1, UCHL5, MKI67IP, FIP1L1, RPL8, MRPL42, ATP5C1, KRR1, RPL15, RPLP0, PUF60, ZRANB2, AIMP1, DDX54, MRPL4, SF3A1, POLR3E, RBM33, THRAP3, GNL3L, RPL4, DDX55, DHX33, RPL3, RPS8, DDX27, ERAL1, RRS1, PRR3, RNPC3, DDX50, RPL32, DDX39A, DDX39A, RPL5, RBFOX2, PPIG, RPS11, THOC4, RRP36, RPS4X, CCNL1, GLTSCR2, RBM46, RBM15, DHX16, CPSF4L, CPSF4, BMS1, BUD13, PRPF3, MRPL39, DKC1, KIAA0020, CLK3, RTN4, RPL23A, DDX18, RPL14, EDF1, CELF4, DDX55, SAMSN1, ZRSR2, DIEXF, RPL15, DHX30, SRSF4, SRP14, ZCRB1, LARP7, CPSF7, ELAVL2, FRG1, RPS24, DAZ3, NAT10, SRSF7, MKI67IP, DND1, DDX47, PA2G4, SRSF8, RPS4X, MRPL3, RSL1D1, PABPC5, NOC2L, APOBEC3D, UTP11L, G3BP2, ACAA2, DDX28, YTHDC1, RBM19, NOP9, SNIP1, RBM28, C1orf35, SFRS10, SFRS17A, CCDC59, RPL27, EIF2C1, NOL7, RBM34, LYAR, RPL28, KRR1, RBMX, PUF60, REXO4, PPAN, CCDC137, ZRSR1, SRSF12, ZRSR2, U2AF1, SRSF5, ADAD2, RBMX2, SRSF10, UTP23, SRSF6, ZC3H18, GPATCH4, HNRNPD, ABT1, TOP3B, HNRNPA1, RPL35, ELAVL4, RBM47, RRP8, RBM15B, PPIL4, ANXA2, RTCA, NOP56, GTF2E2, CPEB4, ZCCHC17, HNRNPR, PIWIL2, EEF2, RNPS1, RDBP, DDX49, RPL35, DDX52, FBL, ZFP36L1, DDX23, NOLC1, RBMX2, HNRNPCL1, TRA2A, HNRNPC, or HERC5.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIGS. 1A-1G show an overview and preliminary testing of experimental design. FIG. 1A shows an exemplary schematic of the dual-luciferase reporter. FIG. 1B shows reporter isoform resulting from distal (top) or proximal (bottom) poly(A) site selection following RBP binding at the MS2 loops. The ratio of each isoform present was measured using the ratio of Renilla to Firefly. FIGS. 1C-1D show exemplary schematics showing the location of the MS2 loops (RBP binding site) relative to the L3 poly(A) site for the downstream (FIG. 1C) and upstream (FIG. 1D) reporters. Also shown are representative data for controls: negative flag (NEG), CPSF5, CPSF6, and HNRNPLCL1. The negative flag represents the poly(A) site usage with no influence from RBP binding. CPSF5, CPSF6 and HNRNPCL1 are previously studied RBPs with known expected effects on poly(A) site selection. FIG. 1E shows an exemplary schematic of the experimental design. The screen was conducted using a 96-well plate format. The ratio of Renilla to Firefly was calculated for each RBP and used to find significant RBPs and the effect and efficiency with which they can regulate APA. FIG. 1F shows an example of results obtained from screening 12 RBPs and controls using the downstream and upstream reporters. FIG. 1G shows results of tethered and untethered assays for downstream and upstream reporters.

FIGS. 2A-2H show an overview of data collected from the screen. FIGS. 2A-2B show normalized mean ratio of Renilla to Firefly for all RBPs tested using the downstream (FIG. 2A) and upstream (FIG. 2B) reporters. Significant RBP candidates that promote distal poly(A) site (PAS) usage are pink, significant RBP candidates that promote proximal PAS are denoted in purple, and candidates that exhibited neither are denoted in yellow. FIGS. 2C-2D show Venn diagrams showing the overlap of RBP candidates bound either upstream or downstream of the PAS and promote proximal PAS (FIG. 2C) or distal PAS (FIG. 2D). FIG. 2E shows Gene Ontology analysis of significant RBP candidates categorized by the effect observed and reporter used. FIG. 2F shows protein domain enrichment analysis of RBP candidates categorized by the effect observed and reporter used. FIG. 2G shows the number of RBP candidates by category that have previously been associated with APA. FIG. 2H shows top ten downstream activators (top) and upstream activators (bottom) ranked by effect.

FIGS. 3A-3B show binding profiles of activator RBP candidates in mammalian cells FIG. 3A shows binding profiles in HEPG2 (top) and K562 (bottom) cells for downstream activator RBP candidates with available data in the ENCODE database. FIG. 3B shows binding profiles in HEPG2 (top) and K562 (bottom) cells for upstream activator RBP candidates with available data in the ENCODE database.

FIGS. 4A-4B show binding profiles of inhibitor RBP candidates. FIG. 4A shows binding profiles in HEPG2 (top) and K562 (bottom) cells for downstream inhibitor RBP candidates with available data in the ENCODE database. FIG. 4B shows binding profiles in HEPG2 (top) and K562 (bottom) cells for upstream inhibitor RBP candidates with available data in the ENCODE database.

FIGS. 5A-5E show an overview of the modalities with which the RBP candidates or enriched protein domains may be paired with to create a molecular tool or RNA therapeutic that modulates RNA by regulating APA. FIG. 5A shows an exemplary schematic of CRISPR-cas entities. FIG. 5B shows an exemplary schematic of CIRTS. FIG. 5C shows an exemplary schematic of ASO. FIG. 5D shows an exemplary schematic of Bifunctional Small molecule. FIG. 5E shows an exemplary schematic of PUF scaffold.

### DETAILED DESCRIPTION

The present disclosure describes methods of regulating gene expression of a target RNA that include delivering an RNA regulation unit into a cell, wherein the RNA regulation unit comprises, consists essentially of, or consists of an RNA binding protein (RBP), and a gene-targeting agent, wherein the RNA binding protein binds proximal to a poly(A) signal, thereby regulating gene expression of the target RNA in the cell.

Various non-limiting aspects of these methods are described herein, and can be used in any combination without limitation. Additional aspects of various components of methods for regulating gene expression are known in the art.

It must be noted that, as used in the specification and the appended claims, the singular forms “a,” “an” and “the” include plural referents unless the context clearly dictates otherwise.

As used herein, the terms “about” and “approximately,” when used to modify an amount specified in a numeric value or range, indicate that the numeric value as well as reasonable deviations from the value known to the skilled person in the art, for example±20%, ±10%, or ±5%, are within the intended meaning of the recited value.

As used herein, a “cell” can refer to either a prokaryotic or eukaryotic cell, optionally obtained from a subject or a commercially available source.

“Comprising” or “comprises” is intended to mean that the compositions, for example media, and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

As used herein, the phrase “gene-targeting agent” refers to an agent that can specifically target an oligonucleotide with a specific nucleic acid sequence. Gene targeting agents can include a CRISPR-Cas system, a CRISPR-cas inspired RNA targeting system (CIRTS), shRNAs, siRNAs, antisense oligonucleotides (ASOs), or microRNA mimics.

As used herein, “delivering”, “gene delivery”, “gene transfer”, “transducing” can refer to the introduction of an exogenous polynucleotide into a host cell, irrespective of the method used for the introduction. Such methods include a variety of well-known techniques such as vector-mediated gene transfer (e.g., viral infection/transfection, or various other protein-based or lipid-based gene delivery complexes) as well as techniques facilitating the delivery of “naked” polynucleotides (e.g., electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

In some embodiments, a polynucleotide can be inserted into a host cell by a gene delivery molecule. Examples of gene delivery molecules can include, but are not limited to, liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

As used herein, the term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “exogenous” refers to any material introduced from or originating from outside a cell, a tissue or an organism that is not produced by or does not originate from the same cell, tissue, or organism in which it is being introduced.

As used herein, the term “expression” refers to the process by which polynucleotides are transcribed into mRNA and/or the process by which the transcribed mRNA is subsequently translated into peptides, polypeptides, or proteins. In some embodiments, if the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, “nucleic acid” is used to include any compound and/or substance that comprise a polymer of nucleotides. In some embodiments, a polymer of nucleotides are referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G).

In some embodiments, the term “nucleic acid” refers to a deoxyribonucleic acid (DNA) or ribonucleic acid (RNA), or a combination thereof, in either a single-or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the isolated nucleic acids described herein, the isolated nucleic acid is DNA. In some embodiments of any of the isolated nucleic acids described herein, the isolated nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine).

Unless otherwise specified, a “nucleotide sequence encoding a protein” includes all nucleotide sequences that are degenerate versions of each other and thus encode the same amino acid sequence.

As used herein, the term “plurality” can refer to a state of having a plural (e.g., more than one) number of different types of things (e.g., a cell, a genomic sequence, a subject, a system, or a protein). In some embodiments, a plurality of genomic sequences can be more than one genomic sequence wherein each genomic sequence is different from each other.

As used herein, the term “subject” is intended to include any mammal. In some embodiments, the subject is cat, a dog, a goat, a human, a non-human primate, a rodent (e.g., a mouse or a rat), a pig, or a sheep.

As used herein, the term “transduced”, “transfected”, or “transformed” refers to a process by which exogenous nucleic acid is introduced or transferred into a cell. A “transduced,” “transfected,” or “transformed” mammalian cell is one that has been transduced, transfected or transformed with exogenous nucleic acid (e.g., a gene delivery vector) that includes an exogenous nucleic acid encoding RNA-binding zinc finger domain).

### Alternative Polyadenylation (APA)

Polyadenylation is the addition of a poly(A) tail to an RNA transcript, typically a messenger RNA (mRNA). The poly(A) tail consists of multiple adenosine monophosphates (i.e., a stretch of RNA with only adenine bases). In some embodiments, polyadenylation is part of a process that produces mature mRNA for translation, therefore, forming part of the larger process of gene expression. The process of polyadenylation begins as the transcription of a gene terminates, wherein the 3′ end of the newly made pre-mRNA is first cleaved off by a set of proteins (e.g., cleavage/polyadenylation specificity factor (CPSF), cleavage stimulation factor (CstF), polyadenylate polymerase (PAP), polyadenylate binding protein 2 (PABII), cleavage factor I (CFI), and cleavage factor II (CFII)). These proteins then synthesize the poly(A) tail at the RNA's 3′ end.

The poly(A) tail is important for the nuclear export, translation and stability of mRNA. The tail is shortened over time, and, when it is short enough, the mRNA is enzymatically degraded. In some embodiments, a gene can have one or more poly(A) tail(s) added at one of several possible sites. In some embodiments, polyadenylation can produce more than one transcript from a single gene (e.g., alternative polyadenylation).

As used herein, “alternative polyadenylation (APA)” refers to a regulatory mechanism that generates diverse 3′ ends on mRNA. In some embodiments, many protein-coding genes can have more than one polyadenylation site, so a gene can code for several mRNAs that differ in their 3′ end. The 3′ region of a transcript can contain many polyadenylation signals (PAS), wherein when more proximal (closer towards 5′ end) PAS sites are utilized, the length of the 3′ untranslated region (3′ UTR) of the transcript is shortened. APA patterns are often tissue specific and play an important role in cellular processes such as cell proliferation, differentiation, and response to stress. In some embodiments, a plurality of APA sites can be found in 3′ UTRs, thereby allowing generation of mRNA isoforms with different 3′ UTR contents. These alternate 3′ UTR isoforms can change how the transcript is regulated, affecting its stability and translation. In some embodiments, since the subcellular localization of a transcript can be regulated by 3′ UTR sequences, APA can also play a role in changing transcript location.

Provided herein are methods of regulating alternative polyadenylation (APA) of a target RNA in a cell, the method including (a) assembling an RNA regulation unit, wherein the RNA regulation unit comprises, consists essentially of, or consists of an RNA binding protein (RBP) and a gene-targeting agent, wherein the RNA binding protein binds proximal to a poly(A) signal; (b) delivering the RNA regulation unit into the cell; and (c) detecting change in the target RNA translation, wherein the RNA regulation unit regulates alternative polyadenylation (APA) of the target RNA in the cell. In some embodiments, regulating APA can in turn, modulate RNA stability, location, and translation of the target RNA.

### RNA Regulation Unit

Another aspect of the disclosure is directed to an RNA regulation unit. As used herein, an “RNA regulation unit” can refer to a system that can recognize specific poly(A) signals and/or sites and regulate alternative polyadenylation (APA) of an RNA. In some embodiments, an RNA regulation unit comprises, consists essentially of, or consists of an RNA binding protein (RBP), and a gene-targeting agent. In some embodiments, the RNA binding protein binds proximal to a poly(A) signal of the RNA. In some embodiments, the RNA binding protein binds to a plurality of poly(A) signals and/or sites of the RNA.

### RNA Binding Protein (RBP)

As used herein, “RNA binding protein” can refer to a protein that interacts with the double or single stranded RNA in cells and participate in forming ribonucleoprotein complexes. RNA binding proteins (RBPs) play a major role in post-transcriptional control of RNAs (e.g., splicing, polyadenylation, mRNA stabilization, mRNA localization, and translation). The term “RNA binding protein” can also refer to a protein that interacts with RNA molecules (e.g., mRNA) from synthesis to decay to affect their metabolism, localization, stability, and translation.

In some embodiments, an RBP is a nuclear protein. In some embodiments, RBPs can include, but are not limited to, splicing factors, RNA stability factors, histone stem-loop binding proteins, or ribosomes. For example, a eukaryotic ribosome can include a collection of RBPs that can interact directly with mRNA coding sequences. In some embodiments, an RBP is a cytoplasmic protein. In some embodiments, an RNA binding protein comprises, consists essentially of, or consists of a ribosomal protein, wherein the ribosomal protein binds to a ribosome and an mRNA during translation. In some embodiments, an RNA binding protein comprises, consists essentially of, or consists of a ribosomal protein, wherein the ribosomal protein binds to a ribosome or an mRNA during translation.

In some embodiments, the RNA binding proteins described herein can include the selection and delivery of an RNA regulation unit comprising an RNA binding protein that binds proximal to a poly(A) signal of the target RNA in a cell. In some embodiments, the RNA binding protein binds upstream of a poly(A) signal. In some embodiments, the RNA binding protein binds downstream of a poly(A) signal. In some embodiments, the RNA binding protein binds a near a proximal poly(A) signal and/or site of the target RNA. In some embodiments, the RNA binding protein binds near a distal poly(A) signal and/or site of the target RNA.

In some embodiments, the RNA binding protein binds proximal to a poly(A) signal of the target RNA. In some embodiments, the RNA binding protein binds to a plurality of proximal poly(A) signals of the target RNA. In some embodiments, the RNA binding proteins binds to a distal to a poly(A) signal of the target RNA. In some embodiments, the RNA binding protein binds to a plurality of distal poly(A) signals of the RNA. In some embodiments, the RNA binding protein binds to a poly(A) signal of the target RNA. In some embodiments, the RNA binding protein binds to a plurality of poly(A) signals of the target RNA. In some embodiments, the RNA binding protein binds to a poly(A) site of the target RNA. In some embodiments, the RNA binding protein binds to a plurality of poly(A) sites of the target RNA.

In some embodiments, a poly(A) signal includes polyadenylation signals that are typically characterized by one of the following sequences: a AATAAA, AAUAAA, ATTAAA, AUUAAA sequence. In some embodiments, poly(A) signals are located downstream of 3′ exons. In some embodiments, poly(A) signals lie within the 5′ untranslated region. In some embodiments, a poly(A) site includes the site of cleavage at which a poly(A) tail is added in mRNA. In some embodiments, a poly(A) site can be determined by comparing cDNA and gDNA. In some embodiments, the sequence at or immediately 5′ to the site of the RNA cleavage is frequently, but not always marked by a “CA”.

Furthermore, RNA-binding proteins (RBPs) have roles in controlling the fate of RNAs including the modulation of pre-mRNA splicing, RNA modification, translation, stability, and localization. RBPs are a group of proteins that interact with RNA using an array of strategies from well-defined RNA-binding domains to disordered regions that recognize RNA sequence and/or secondary structures.

In some embodiments, the RBP is an upstream activator of a polyadenylation site. In some embodiments, the RNA binding protein comprises, consists essentially of, or consists of CPSF5, RNPS1, CPSF6, CSTF1, RBM11, TRNAU1AP, RBM14, MBNL1, PRRC2B, EIF4B, LGALS1, LUC7L, APOBEC3A, FUBP1, CDC40, UBE2I, SRP68, NGRN, ZRANB2, GRB2, RBM5, ZC3H18, PRPF40A, TIAL1, RBM10, ZC3HAV1, RPS10, YTHDF1, EIF4A3, IGF2BP3, SAMD4A, PNN, CLK2, PRPF4, RPS28, EIF4H, RY1, LARP4B, EIF3G, FLYWCH2, CIR1, WDR6, SMNDC1, SLBP, GTSF1, U2AF2, PRPF4, RBBP6, SRSF8, MBNL2, SRSF9, PCBP1, SBDS, PPIA, RPS19BP1, ISY1, CPSF6, DNAJC17, TOB2, RBM38, RPS21, LGALS3, CPSF7, DDX6, TRA2A, BUD13, SNRPA, STXBP1, RPL30, LSM1, GNB2L1, SARNP, HFM1, CNOT3, METTL16, LSM5, CCDC75, CHAF1B, STAU1, AHNAK, RBM8A, MAZ, NOSIP, PARN, SPATS2L, SNRNP40, YWHAG, LSM10, SNRNP70, PHF5A, RBFOX1, TPD52L2, RBM18, BCDIN3D, U2AF1, CSTF2T, SFPQ, SUPT5H, LARP4, EDC3, ASCC1, MKI67IP, EIF4A1, PDIA3, SSRP1, HNRNPCL1, PAPOLG, EDF1, RBM12B, DCD.

In some embodiments, the RBP is an upstream inhibitor of a polyadenylation site. In some embodiments, the RNA binding protein comprises, consists essentially of, or consists of RBPMS, RPL23, DHX16, NDUFV3, LUC7L3, RPL31, SRSF3, RPP21, SUMO1, FKBP4, FASN, DDX39A, EZR, RBFOX2, ZCRB1, RAN, AHNAK, SRSF11, SERBP1, PIWIL4, FUBP3, ERCC3, EIF2C2, DNAJC2, RBM26, NUDT16L1, SLIRP, APOBEC3C, LSMD1, SRBD1, RPS3A, SSBP1, WDR3, RPL36, SUGP1, STIP1, SLC3A2, IFIT2, RPL23, ELMOD3, CPSF1, APOBEC3G, GLRX3, XPO5, COA6, CPSF4, HSPD1, PTRF, DCP2, XPO5, RPS15A, DDX20, GRN, PHF6, SUGP2, RBMS1, SF1, RBM33, SRPR, NAA15, DCN, DIS3, SF3B2, TRAP1, MTPAP, FTO, C1orf131, RPL3, IFI16, USP36, RBM25, EXOSC3, NPM1, MRPS5, CCDC9, SEC61B, ZCCHC7, FAM120A, DNTTIP2, NOLC1, EIF2S2, SAFB2, SNRPE, ILF2, RDBP, THOC5, DDX1, MRPS11, PUS7L, RPL6, NPM1, SF3B4, CLP1, TRIM56, CALR, DHX58, RPS6, ARL6IP4, PRPF38B, APOBEC3B, REPIN1, RBM45, MEPCE, CNOT6, EIF2C2, DMGDH, NOA1, ASS1, RPS3, EIF5B, RBM17, EXOSC10, NSA2, DCP1A, SART3, APEH, TEFM, BTG4, BCCIP, PNLDC1, EIF3H, HSP90AA1, TSR1, CLASRP, CPNE3, PSMD4, FAM98A, PUF60, G3BP1, PRPF19, RPS6, RBFOX2, CPEB1, SEC63, RPL15, NHP2, ZCCHC11, HSPA9, RTN4, NONO, ZCCHC7, EIF2AK2, RPL21, DHX40, ELAVL4, TUFM, RPL5, DHX8, SRFBP1, RPS20, SNRPG, PWP2, THOC3, SUCLG1, PDIA4, ZMAT3, ZYX, DHX36, FAM98A, DDX19B, ANXA2, POLK, FTSJ3, LARP1B, ANXA2, C1D, ACAA2, PSMC1, DDX18, PPP1CB, PSPC1, LARP4, CNOT10, FANCM, MRPL11, DDX27, DDX11, SEC63, HEATR1, NOP2, RPS7, DYNC1LI1, SAMSN1, DHX35, EIF3L, PABPN1L, RRS1, HADHB, NUDT16, SLC7A9, CPSF4, MRPL37, ERAL1, RPL8, RNMTL1, SRP14, PPAPDC2, NPM3, CORO1A, STAU2, RPL28, CELF4, RPUSD4, YTHDC1, DIMT1, HADHB, TBRG4, DDX55, FASTK, ALDH6A1, CPSF2, SF3B3, SNRPB2, PUS7, RPL35, DDX53, MDH2, FXR2, ILF3, METTL3, TRMT2A, HSPA8, SF3A1, TPT1, FDPS, CLK3, RRP8, RPS8, GPANK1, LARP6, TDRD9, TARDBP, ATP5C1, MRPS11, PCBP4, RBM26, UTP11L, RPL4, CCDC137, PINX1, RNMTL1, FZD8, NAP1L3, POLR2G, DDX31, RPS2, DDX41, SRPR, RBM28, RPN1, RPS24, NXF1, G3BP2, U2AF1, RTCA, RBM46, BYSL, DYNC1H1, PRR3, POLR3E, RBM6, PUF60, RTN4, GANAB, TSR1, TRUB2, DDX28, TERT, DIEXF, NOP58, APOBEC3D, IFIH1, QKI, KIAA1324, EEF1A1, RALY, CNOT7, RPS4X, DDX39A, FASTKD2, NOL10, DDX11, PES1, EFTUD2, PPIL4, RSL1D1, SNRPN, KRR1, MRPL32, ELAVL2, NANOS3, CIRBP, CCDC59, DDX49, ZFC3H1, RPL15, ALDH18A1, HERC5, MRM1, CNBP, RPLP0, KRT18, TOP3B, FSCN1, SLC25A5, SURF6, CPSF4L, RPL14, MRPL39, THOC6, PKM, RPL3, NOL7, DDX43, GSPT2, NOP16, NOL12, RBM12, ABT1, KRR1, HTATSF1, KIAA0020, NOP16, RPL27, UTP23, BST2, RBM47, EIF2C1, MAK16, PTCD1, DDX60, GPATCH4, PA2G4, DDX55, LARP7, GTF2E2, USP32, THUMPD1, MRPL30, DDX59, MRPS24, UCHL5, NXF3, AIMP1, THOC4, GLTSCR2, DHX33, KHDRBS2, BMS1, DDX1, NOC2L, MAP4, SAMD4A, YARS, RBM34, HDGF, PIWIL1, TRMU, ZFP36L1, TROVE2, ACTN4, RBM19, AK8, XRCC6, FAM208A, SRSF10, NAT10, SNRNP35, RPL7, MRPL3, NOP9, ARL6IP4, DDX52, HNRNPD, MRPS23, RPL15, HSP90AB1, RBM4, DHX30, HNRNPA1, NXF2, THRAP3, ADAD2, RPL13A, RPL35, HMGB2, HIST1H1C, EIF2C3, HNRNPC, ILF3, RBM15B, RPL19, MRPL4, DUS2L, TRIP6, GTPBP4, EEF2, DDX54, RPL23A, RRP36, PPAN, PATL1, REXO4, DND1, NOP56, RPS11, RBM15, RPS4X, MRPS15, HNRNPR, PIWIL2, SYNCRIP, SNRPB, RPL32, FBL.

In some embodiments, the RBP is a downstream activator of a polyadenylation site. In some embodiments, the RNA binding protein comprises, consists essentially of, or consists of CSTF1, TOB2, PRRC2B, PARN, RBM11, DDX17, GTSF1, DDX5, RBM10, SNRPA, RBM22, CNOT4, RBM14, MBNL1, SUPT5H, ZC3HAV1, RBM4, CNOT2, SAMD4A, CDC40, HNRNPH1, SRP68, EIF4B, CSTF2T, LGALS3, NANOS3, SFPQ, CIR1, PPP1CA, ZMAT3, PRPF4, PRPF4, SCAF8, TRNAU1AP, EDC3, PCBP3, LSM1, PPIA, TPD52L2, RBM5, APOBEC3A, LGALS1, CDC42EP4, DZIP3, HNRNPH2, STAU2, PCBP1, GRB2, NUP35, EIF4H, BTG1, WDR6, THUMPD1, RSRC1, SMNDC1, SYMPK, NONO, MBNL2, MAZ, STAU1, HNRNPF, TFIP11, SMN1, AHNAK, YTHDF1, NGRN, METTL16, SNRPG, SF3A2, STAU2, YWHAE, YWHAG, SFRS2IP, PEG10, HRSP12, EXOSC10, CDC42EP4, LSM7, ZC3H7A, SNRPN, RBM42, AHNAK, GSPT1, SMN1, MECP2, SRSF9, QARS, ASCC3, CUGBP1, ZFC3H1, NMD3, SNRPB, SRSF2, NUSAP1, RBM38, CORO1A, SFRS17A, BTG4, GTPBP10, SNW1, CPSF6.

In some embodiments, the RBP is a downstream inhibitor of a polyadenylation site. In some embodiments, the RNA binding protein comprises, consists essentially of, or consists of FZD3, PPIG, CSTF3, WDR36, DHX8, SLIRP, CNOT10, SOX21, MRPS11, PURG, ADK, TRAP1, LSM4, NGDN, DYNC1H1, FLYWCH2, NPM1, IGF2BP1, ASS1, GNL2, RBPMS, LSM10, FAM46A, TPT1, RNMTL1, LARP4, CPSF5, LUC7L3, NAA15, RBM3, TPT1, ELAC2, RPGR, PNO1, UTP3, SNRPB2, HIST1H1C, ASCC1, SART3, EIF2C3, EIF4A1, EIF3L, MRPS15, LUC7L, SNRPE, LIN28A, CNOT10, NHP2, PARP12, HADHB, MRPS30, XPO5, RPS15A, NPM1, PURB, MTPAP, MRPL11, COA6, GLE1, NUDT16, GRN, RBM17, DHX57, PHF5A, SMG6, SARNP, SUGP2, TSR1, KRR1, C1D, ESRP1, PSMD4, SRSF10, TRMT10C, ZGPAT, BOLL, SPATS2L, MRPS24, RBM26, RPL6, CLASRP, DDX59, SEC63, EIF2S2, RPL23, DHX37, CPEB1, SNRPF, MRPS31, PNLDC1, MRPS23, RPS3A, TEFM, CCT4, POLR2G, RPS7, CNBP, FUBP1, SRPR, DDX60, NAP1L3, RPL22, EIF3C, DDX27, NOA1, LARP1B, FDPS, USP36, CMSS1, CCNL2, RPP30, SF3B3, MEPCE, SBDS, RPUSD4, U2AF2, RPL21, THOC3, FXR2, RPL6, LSM6, FAM98A, WDR33, DUS2L, THOC5, TRIM39, FAM208A, RRP7A, MRPS35, P4HB, LARP1, MAK16, RBM4, EIF2C2, ARL6IP4, FZD8, FTSJ3, PUS7, IFIT2, EIF2C2, S100A4, DHX58, TRIM56, SRPR, SERBP1, MRPL30, GTF2F1, LSMD1, TFB1M, FKBP3, PHF6, METTL3, ALDH18A1, ZFC3H1, GSPT2, GTPBP4, EXOSC3, PRPF6, RC3H2, NSUN2, DHX35, DCN, ANXA2, DHX40, SAFB2, RPS6, IFIH1, DDX43, NOL12, GFM1, RPS20, PUF60, PIWIL1, RBM8A, LARP6, HSP90AB1, MRPS5, MAP4, MRPL37, PRPF19, XRN2, HFM1, ALDH6A1, FTO, SPATS2, TRMU, USP32, TROVE2, RPS15A, XRCC6, DNAJC17, NOP58, RPL7, RPL10A, DDX1, C16orf88, APOBEC3B, PPAPDC2, HDGF, C1orf131, SUPV3L1, RPS3, TDRD9, RALYL, CPSF2, SUCLG1, DDX20, RPN1, RPP25, PTRH1, EEF1A1, FAM32A, SEC63, CNOT6, ILF3, SRFBP1, TRIP6, NOSIP, FSCN1, NOB1, RPS6, GNL2, SRPK2, EIF3D, RPL15, TRMT6, RPL8, NOP2, CHD2, CIRBP, RBMS1, SERBP1, NOP16, FASTKD2, CPSF4, DDX11, CHTOP, PWP2, SREK1, LSM3, BCCIP, BYSL, SECISBP2, TRMT2A, REPIN1, CIRH1A, MRTO4, RPL19, HADHB, NOL10, SYF2, MFAP1, APOBEC3C, PRPF38B, RPS2, NSA2, CPSF3L, SNRNP35, THOC6, DDX11, RPL13A, DDX31, NPM1, BST2, PRPF40A, PES1, UCHL5, MKI67IP, FIP1L1, RPL8, MRPL42, ATP5C1, KRR1, RPL15, RPLP0, PUF60, ZRANB2, AIMP1, DDX54, MRPL4, SF3A1, POLR3E, RBM33, THRAP3, GNL3L, RPL4, DDX55, DHX33, RPL3, RPS8, DDX27, ERAL1, RRS1, PRR3, RNPC3, DDX50, RPL32, DDX39A, DDX39A, RPL5, RBFOX2, PPIG, RPS11, THOC4, RRP36, RPS4X, CCNL1, GLTSCR2, RBM46, RBM15, DHX16, CPSF4L, CPSF4, BMS1, BUD13, PRPF3, MRPL39, DKC1, KIAA0020, CLK3, RTN4, RPL23A, DDX18, RPL14, EDF1, CELF4, DDX55, SAMSN1, ZRSR2, DIEXF, RPL15, DHX30, SRSF4, SRP14, ZCRB1, LARP7, CPSF7, ELAVL2, FRG1, RPS24, DAZ3, NAT10, SRSF7, MKI67IP, DND1, DDX47, PA2G4, SRSF8, RPS4X, MRPL3, RSL1D1, PABPC5, NOC2L, APOBEC3D, UTP11L, G3BP2, ACAA2, DDX28, YTHDC1, RBM19, NOP9, SNIP1, RBM28, C1orf35, SFRS10, SFRS17A, CCDC59, RPL27, EIF2C1, NOL7, RBM34, LYAR, RPL28, KRR1, RBMX, PUF60, REXO4, PPAN, CCDC137, ZRSR1, SRSF12, ZRSR2, U2AF1, SRSF5, ADAD2, RBMX2, SRSF10, UTP23, SRSF6, ZC3H18, GPATCH4, HNRNPD, ABT1, TOP3B, HNRNPA1, RPL35, ELAVL4, RBM47, RRP8, RBM15B, PPIL4, ANXA2, RTCA, NOP56, GTF2E2, CPEB4, ZCCHC17, HNRNPR, PIWIL2, EEF2, RNPS1, RDBP, DDX49, RPL35, DDX52, FBL, ZFP36L1, DDX23, NOLC1, RBMX2, HNRNPCL1, TRA2A, HNRNPC, HERC5.

Each of the RBP genes recited in this disclosure is well known in the art and described in Genecards® (genecards.org) and NCBI Protein (ncbi.nlm.nih.gov/protein).

### Gene-Targeting Agent

As used herein, the term “gene-targeting agent” can refer to an agent that targets the RNA binding protein to the target RNA (e.g., mRNA). In some embodiments, the gene-targeting agent can include a programmable RNA-targeting platform. As used herein, a “programmable RNA-targeting platform” refers to a system of targeting RNA wherein the targeting entity is an RNA molecule that can be engineered to specifically target an RNA of choice. In some embodiments, the gene-targeting agent can include a non-Cas RNA-targeting system. In some embodiments, the gene-targeting agent can include CRISPR-Cas inspired RNA targeting systems (CIRTS). In some embodiments, the gene-targeting agent can include RNA interference (e.g., short hairpin RNA (shRNA), small interfering RNA (siRNA), antisense oligonucleotide (ASO), or microRNA mimics). In some embodiments, the gene-targeting agent can include small molecules that is able to target a three-dimensional structure on a target RNA and recruit a select endogenous protein (e.g., RNA binding protein). In some embodiments, the gene-targeting agent comprises, consists essentially of, or consists of a non-guided RNA-binding polypeptide that is capable of binding a target RNA without a corresponding gRNA sequence. In some embodiments, the non-guided RNA-binding polypeptide can include a PUF protein (Pumilio and FBF homology family). In some embodiments, the gene-targeting agent can include an engineered protein domain. In some embodiments, the engineered protein domain can include a PUF domain.

### CRISPR-Cas Systems

In some embodiments, the gene-targeting agent can include CRISPR components. For example, in some embodiments, CRISPR components can include, but are not limited to, a guide RNA and a CRISPR-associated endonuclease (Cas protein). In some embodiments, the gene-targeting agent can include a guide RNA (e.g., gRNA or sgRNA) and a CRISPR-associated endonuclease (Cas protein). In some embodiments, the gene-targeting agent comprises, consists essentially of, or consists of shRNAs, siRNAs, ASOs, or microRNa mimics. In some embodiments, the gene-targeting agent can include a Cas RNA targeting system. In some embodiments, the Cas RNA targeting system includes an inactive Cas protein. In some embodiments, the inactive Cas protein is an inactive Cas9 (dCas9). In some embodiments, the inactive Cas protein is an inactive Cas13 (dCas13).

As used herein, the term “CRISPR” refers to a technique of sequence specific genetic manipulation relying on the clustered regularly interspaced short palindromic repeats pathway, which unlike RNA interference regulates gene expression at a transcriptional level. The term “gRNA” or “guide RNA” refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32(12):1262-7 and Graham, D., et al. Genome Biol. 2015; 16:260, both of which are incorporated herein in their entireties.

In some embodiments, the guide RNA can recognize a target RNA, for example, by hybridizing to the target RNA. In some embodiments, the guide RNA comprises, consists essentially of, or consists of a sequence that is complementary to the target RNA. In some embodiments, the gRNA can include one or more modified nucleotides. In some embodiments, the gRNA has a length that is about 10 nt (e.g., about 20 nt, about 30 nt, about 40 nt, about 50 nt, about 60 nt, about 70 nt, about 80 nt, about 90 nt, about 100 nt, about 120 nt, about 140 nt, about 160 nt, about 180 nt, about 200 nt, about 300 nt, about 400 nt, about 500 nt, about 600 nt, about 700 nt, about 800 nt, about 900 nt, about 1000 nt, or about 2000 nt).

In some embodiments, a guide RNA can recognize a variety of RNA targets. For example, a target RNA can be messenger RNA (mRNA), ribosomal RNA (rRNA), signal recognition particle RNA (SRP RNA), transfer RNA (tRNA), small nuclear RNA (snRNA), small nucleolar RNA (snoRNA), antisense RNA (aRNA), long noncoding RNA (IncRNA), microRNA (miRNA), piwi-interacting RNA (piRNA), small interfering RNA (siRNA), short hairpin RNA (shRNA), retrotransposon RNA, viral genome RNA, or viral noncoding RNA. In some embodiments, a target RNA can be an RNA involved in pathogenesis of conditions such as cancers, neurodegeneration, cutaneous conditions, endocrine conditions, intestinal diseases, infectious conditions, neurological conditions, liver diseases, heart disorders, or autoimmune diseases. In some embodiments, a target RNA can be a therapeutic target for conditions such as cancers, neurodegeneration, cutaneous conditions, endocrine conditions, intestinal diseases, infectious conditions, neurological conditions, liver diseases, heart disorders, or autoimmune diseases.

### CRISPR-Cas Inspired RNA Targeting Systems

In some embodiments, the gene-targeting agent can include CRISPR-Cas inspired RNA targeting systems (CIRTS). Similar to CRISPR/Cas-based systems, CIRTS is a ribonucleoprotein complex that uses Watson-Crick-Franklin base pair interactions to deliver protein cargo site-selectively in the transcriptome. CIRTS can be engineered to deliver a range of regulatory proteins to transcripts, including nucleases for degradation, deadenylation regulatory machinery for degradation, or translational activation machinery for enhanced protein production. However, CIRTS are up to 5-fold smaller than most CRISPR/Cas systems and can be engineered entirely from human parts.

CIRTS can include an RNA regulatory system or method of at least one of each: i) an RNA hairpin binding domain; ii) an RNA targeting molecule comprising an RNA targeting region and at least one hairpin structure, wherein the hairpin structure of the RNA targeting molecule specifically binds to i; and iii) an RNA regulatory domain. In some embodiments, the following are included: i) and ii), i) and iii), ii) and iii), or i), ii), and iii). Any embodiment disclosed herein can contain any of these combinations.

Additional description of CRISPR-Cas inspired RNA targeting systems include those described in US2022/0048962, which is incorporated herein by reference in its entirety.

### RNA Interference

In some embodiments, the gene-targeting agent can include systems of RNA interference. Exemplary RNAi molecules can be, without limitation, shRNA, siRNA, piwi-interacting RNA (piRNA), micro RNA (miRNA), double-stranded RNA (dsRNA), antisense RNA, or any other RNA species that can be cleaved inside a cell to form interfering RNAs.

As used herein, siRNAs include, without limitation, modified siRNAs, including siRNAs with enhanced stability in vivo. Modified siRNAs include molecules containing nucleotide analogues, including those molecules having additions, deletions, and/or substitutions in the nucleobase, sugar, or backbone; and molecules that are cross-linked or otherwise chemically modified. (See Crooke, U.S. Pat. Nos. 6,107,094 and 5,898,031; Elmen et al., U.S. Publication Nos. 2008/0249039 and 2007/0191294; Manoharan et al., U.S. Publication No. 2008/0213891; MacLachlan et al., U.S. Publication No. 2007/0135372; and Rana, U.S. Publication No. 2005/0020521; all of which are hereby incorporated by reference.) As used herein, the term siRNA can refer to siRNA molecules that are produced in vitro, and then introduced into a cell. In some aspects, as used herein, an siRNA molecule is not limited to naturally occurring nucleotides, and can incorporate any one or plurality of unnatural structures or chemical modifications, generally where the use of such unnatural structures or modifications result in an siRNA molecule with improved activity or stability.

Another example of RNA interference (RNAi) includes short hairpin RNAs (shRNAs). shRNAs consist of a stem-loop structure that can be transcribed in cells from an RNA polymerase II or RNA polymerase III promoter on a plasmid construct. It has been shown that expression of shRNA from a plasmid can be stably integrated for constitutive expression. shRNAs are synthesized in the nucleus of cells, further processed and transported to the cytoplasm. In some embodiments, the shRNA comprises, consists essentially of, or consists of a stem region and a loop region. In some embodiments, the stem region comprises, consists essentially of, or consists of a double-stranded (duplex) region of base paired nucleotides. The duplex region can comprise from 19 to 29 base pairs. The base pairs can be contiguous or non-contiguous. In a certain embodiment, the duplex region contains 29 contiguous or non-contiguous base pairs. The loop region is useful at 3 to 23 nucleotides in length.

Another example of RNAi includes micro-RNA (miRNA), an endogenous RNA interference molecule that is synthesized in the nucleus in a form mirrored by shRNA. This class of short, single-stranded miRNAs are found both in plant and animal cells, and are derived from larger precursors that form a predicted RNA stem-loop structure. These miRNA precursor molecules are transcribed from autonomous promoters—or are instead contained within longer RNAs. More than 300 distinct miRNAs have been discovered to date, some of which have been found to be expressed in organisms as diverse as nematodes. miRNAs appear to play a role in the regulation of gene expression, primarily at the post-transcriptional level via translation repression. Like mRNAs, miRNAs are initially transcribed by RNA polymerase II into a long primary transcript (pri-miRNA) that contains one or more hairpin-like stem-loop shRNA structures. The stem-loop shRNA structures within the pri-miRNA are further processed in the nucleus by the RNase III enzyme Drosha and its cofactor DGCR-8 into pre-miRNA. Pre-miRNA is transported to the cytoplasm by the transport receptor complex Exportin-5-RanGTP, where it interacts with a second RNase III enzyme Dicer and its cofactor TRBP. Dicer trims off the loop and presents the remaining double stranded stem to the RISC to target the target RNA.

Another example of RNAi includes antisense oligonucleotides (ASOs), an oligomeric nucleotide that is at least partially complementary to a target nucleic acid molecule to which it hybridizes. In certain embodiments, an antisense oligonucleotide modulates (increases or decreases) expression of a target nucleic acid. Antisense oligonucleotides include, but are not limited to, compounds that are oligonucleotides, oligonucleosides, oligonucleotide analogs, oligonucleotide mimetics, and chimeric combinations.

### Non-Guided RNA-Binding Polypeptides

In some embodiments, the gene-targeting agent can include non-guided RNA-binding polypeptides. In some embodiments, the non-guided RNA-binding polypeptide can include a PUF protein (Pumilio and FBF homology family).

One example of a non-guided RNA-binding polypeptide are PUF proteins, which as used herein, encompass all related proteins and domains of such proteins (which may also be termed PUM proteins), for instance human Pumilio homolog 1 (PUM1), PUM×2 or PUFx2 which are duplicates of PUM1. PUF proteins are typically characterized by the presence of eight consecutive PUF repeats, each of approximately 40 amino acids, often flanked by two related sequences, Csp1 and Csp2. Each repeat has a “core consensus” containing aromatic and basic residues. In some embodiments, the entire cluster of PUF repeats is required for RNA binding. Furthermore, PUF proteins are examples of releasable nucleic acid-binding domains which bind to RBPs, thereby enabling a releasable, reversible attachment of the PUF protein to the RBP. PUF proteins are found in most eukaryotes and is involved in embryogenesis and development. PUFs has one domain that binds RNA that is composed of 8 repeats generally containing 36 amino acids, which is the domain typically utilized for RNA binding. Each repeat binds a specific nucleotide and it is commonly the amino acid in position 12 and 16 that confer the specificity with a stacking interaction from amino acid 13. The naturally occurring PUFs can bind the nucleotides adenosine, uracil and guanosine, and engineered PUFs can also bind the nucleotide cytosine. Hence the system is modular and the 8-nucleotide sequence that the PUF domain binds to can be changed by switching the binding specificity of the repeat domains. Hence, the PUF proteins can be natural or engineered to bind to a target RNA molecule. There are also engineered and/or duplicated PUF domains that bind 16-nucleotides in a sequence-specific manner, which can also be utilized to increase the specificity to the target RNA. Hence the PUF domain can be modified to bind any sequence, with different affinity and sequence length, which make the system highly modular and adaptable. The PUF binding site on the target RNA is typically longer than the sequence bound by many other RNA-binding proteins, and can include 5 nucleotides (nt), 6 nt, 7 nt, 8 nt, 9 nt, 10 nt, 11 nt, 12 nt, 13 nt, 14 nt, 15 nt, 16 nt, 17 nt, 18 nt, 19 nt, or even 20 nt and longer, depending on the need for modifiable sequence specificity of the NA-binding domain.

### Methods of Regulating Alternative Polyadenylation (APA)

Provided herein are methods of regulating alternative polyadenylation (APA) of a target RNA in a cell, the method including (a) assembling an RNA regulation unit, wherein the RNA regulation unit comprises, consists essentially of, or consists of an RNA binding protein (RBP) and a gene-targeting agent, wherein the RNA binding protein binds proximal to a poly(A) signal; (b) delivering the RNA regulation unit into the cell; and (c) detecting change in the target RNA translation, wherein the RNA regulation unit regulates alternative polyadenylation (APA) of the target RNA in the cell. In some embodiments, the target RNA is an endogenous mRNA. In some embodiments, the target RNA is a non-coding RNA.

### Selection of RBP

In some embodiments, the RBPs described herein can be selected according to the goals for the target RNA. For example, non-limiting goals include increasing stability of a target RNA in a cell, preventing degradation of a target RNA in a cell, modifying localization of a target RNA in a cell, and/or increasing synthesis of a protein encoded by a target RNA in a cell. In the methods described herein, the RBP can be selected based on whether that particular RBP has been identified as activating or inhibiting alternative polyadenylation of the target RNA.

In some embodiments, the methods described herein can include the selection and delivery of an RNA regulation unit comprising an RNA binding protein that binds proximal to a poly(A) signal of the target RNA in a cell. In some embodiments, the RNA binding protein binds upstream of a poly(A) signal. In some embodiments, the RNA binding protein binds downstream of a poly(A) signal. In some embodiments, the RNA binding protein binds a near a proximal poly(A) signal and/or site of the target RNA. In some embodiments, the RNA binding protein binds near a distal poly(A) signal and/or site of the target RNA.

In some embodiments, the methods described herein can include the selection and delivery of an RNA regulation unit comprising an RNA binding protein that binds proximal to a poly(A) signal of the target RNA. In some embodiments, the RNA binding protein binds to a plurality of proximal poly(A) signals of the target RNA. In some embodiments, the RNA binding proteins binds to a distal to a poly(A) signal of the target RNA. In some embodiments, the RNA binding protein binds to a plurality of distal poly(A) signals of the RNA. In some embodiments, the RNA binding protein binds to a poly(A) signal of the target RNA. In some embodiments, the RNA binding protein binds to a plurality of poly(A) signals of the target RNA. In some embodiments, the RNA binding protein binds to a poly(A) site of the target RNA. In some embodiments, the RNA binding protein binds to a plurality of poly(A) sites of the target RNA.

In some embodiments, the RNA binding protein binds upstream of a poly(A) signal of the target RNA, and wherein the RBP activates usage of a given proximal poly(A) signal and/or site of the target RNA. In some embodiments, the RNA binding protein comprises, consists essentially of, or consists of CPSF5, RNPS1, CPSF6, CSTF1, RBM11, TRNAU1AP, RBM14, MBNL1, PRRC2B, EIF4B, LGALS1, LUC7L, APOBEC3A, FUBP1, CDC40, UBE2I, SRP68, NGRN, ZRANB2, GRB2, RBM5, ZC3H18, PRPF40A, TIAL1, RBM10, ZC3HAV1, RPS10, YTHDF1, EIF4A3, IGF2BP3, SAMD4A, PNN, CLK2, PRPF4, RPS28, EIF4H, RY1, LARP4B, EIF3G, FLYWCH2, CIR1, WDR6, SMNDC1, SLBP, GTSF1, U2AF2, PRPF4, RBBP6, SRSF8, MBNL2, SRSF9, PCBP1, SBDS, PPIA, RPS19BP1, ISY1, CPSF6, DNAJC17, TOB2, RBM38, RPS21, LGALS3, CPSF7, DDX6, TRA2A, BUD13, SNRPA, STXBP1, RPL30, LSM1, GNB2L1, SARNP, HFM1, CNOT3, METTL16, LSM5, CCDC75, CHAF1B, STAU1, AHNAK, RBM8A, MAZ, NOSIP, PARN, SPATS2L, SNRNP40, YWHAG, LSM10, SNRNP70, PHF5A, RBFOX1, TPD52L2, RBM18, BCDIN3D, U2AF1, CSTF2T, SFPQ, SUPT5H, LARP4, EDC3, ASCC1, MKI67IP, EIF4A1, PDIA3, SSRP1, HNRNPCL1, PAPOLG, EDF1, RBM12B, or DCD.

In some embodiments, the RNA binding protein binds upstream of a poly(A) signal of the target RNA, and wherein the RBP inhibits usage of a given proximal poly(A) signal and/or site of the target RNA. In some embodiments, the RNA binding protein comprises, consists essentially of, or consists of RBPMS, RPL23, DHX16, NDUFV3, LUC7L3, RPL31, SRSF3, RPP21, SUMO1, FKBP4, FASN, DDX39A, EZR, RBFOX2, ZCRB1, RAN, AHNAK, SRSF11, SERBP1, PIWIL4, FUBP3, ERCC3, EIF2C2, DNAJC2, RBM26, NUDT16L1, SLIRP, APOBEC3C, LSMD1, SRBD1, RPS3A, SSBP1, WDR3, RPL36, SUGP1, STIP1, SLC3A2, IFIT2, RPL23, ELMOD3, CPSF1, APOBEC3G, GLRX3, XPO5, COA6, CPSF4, HSPD1, PTRF, DCP2, XPO5, RPS15A, DDX20, GRN, PHF6, SUGP2, RBMS1, SF1, RBM33, SRPR, NAA15, DCN, DIS3, SF3B2, TRAP1, MTPAP, FTO, C1orf131, RPL3, IFI16, USP36, RBM25, EXOSC3, NPM1, MRPS5, CCDC9, SEC61B, ZCCHC7, FAM120A, DNTTIP2, NOLC1, EIF2S2, SAFB2, SNRPE, ILF2, RDBP, THOC5, DDX1, MRPS11, PUS7L, RPL6, NPM1, SF3B4, CLP1, TRIM56, CALR, DHX58, RPS6, ARL6IP4, PRPF38B, APOBEC3B, REPIN1, RBM45, MEPCE, CNOT6, EIF2C2, DMGDH, NOA1, ASS1, RPS3, EIF5B, RBM17, EXOSC10, NSA2, DCP1A, SART3, APEH, TEFM, BTG4, BCCIP, PNLDC1, EIF3H, HSP90AA1, TSR1, CLASRP, CPNE3, PSMD4, FAM98A, PUF60, G3BP1, PRPF19, RPS6, RBFOX2, CPEB1, SEC63, RPL15, NHP2, ZCCHC11, HSPA9, RTN4, NONO, ZCCHC7, EIF2AK2, RPL21, DHX40, ELAVL4, TUFM, RPL5, DHX8, SRFBP1, RPS20, SNRPG, PWP2, THOC3, SUCLG1, PDIA4, ZMAT3, ZYX, DHX36, FAM98A, DDX19B, ANXA2, POLK, FTSJ3, LARP1B, ANXA2, C1D, ACAA2, PSMC1, DDX18, PPP1CB, PSPC1, LARP4, CNOT10, FANCM, MRPL11,DDX27, DDX11, SEC63, HEATR1, NOP2, RPS7, DYNC1LI1, SAMSN1, DHX35, EIF3L, PABPN1L, RRS1, HADHB, NUDT16, SLC7A9, CPSF4, MRPL37, ERAL1, RPL8, RNMTL1, SRP14, PPAPDC2, NPM3, CORO1A, STAU2, RPL28, CELF4, RPUSD4, YTHDC1, DIMT1, HADHB, TBRG4, DDX55, FASTK, ALDH6A1, CPSF2, SF3B3, SNRPB2, PUS7, RPL35, DDX53, MDH2, FXR2, ILF3, METTL3, TRMT2A, HSPA8, SF3A1, TPT1, FDPS, CLK3, RRP8, RPS8, GPANK1, LARP6, TDRD9, TARDBP, ATP5C1, MRPS11, PCBP4, RBM26, UTP11L, RPL4, CCDC137, PINX1, RNMTL1, FZD8, NAP1L3, POLR2G, DDX31, RPS2, DDX41, SRPR, RBM28, RPN1, RPS24, NXF1, G3BP2, U2AF1, RTCA, RBM46, BYSL, DYNC1H1, PRR3, POLR3E, RBM6, PUF60, RTN4, GANAB, TSR1, TRUB2, DDX28, TERT, DIEXF, NOP58, APOBEC3D, IFIH1, QKI, KIAA1324, EEF1A1, RALY, CNOT7, RPS4X, DDX39A, FASTKD2, NOL10, DDX11, PES1, EFTUD2, PPIL4, RSL1D1, SNRPN, KRR1, MRPL32, ELAVL2, NANOS3, CIRBP, CCDC59, DDX49, ZFC3H1, RPL15, ALDH18A1, HERC5, MRM1, CNBP, RPLP0, KRT18, TOP3B, FSCN1, SLC25A5, SURF6, CPSF4L, RPL14, MRPL39, THOC6, PKM, RPL3, NOL7, DDX43, GSPT2, NOP16, NOL12, RBM12, ABT1, KRR1, HTATSF1, KIAA0020, NOP16, RPL27, UTP23, BST2, RBM47, EIF2C1, MAK16, PTCD1, DDX60, GPATCH4, PA2G4, DDX55, LARP7, GTF2E2, USP32, THUMPD1, MRPL30, DDX59, MRPS24, UCHL5, NXF3, AIMP1, THOC4, GLTSCR2, DHX33, KHDRBS2, BMS1, DDX1, NOC2L, MAP4, SAMD4A, YARS, RBM34, HDGF, PIWIL1, TRMU, ZFP36L1, TROVE2, ACTN4, RBM19, AK8, XRCC6, FAM208A, SRSF10, NAT10, SNRNP35, RPL7, MRPL3, NOP9, ARL6IP4, DDX52, HNRNPD, MRPS23, RPL15, HSP90AB1, RBM4, DHX30, HNRNPA1, NXF2, THRAP3, ADAD2, RPL13A, RPL35, HMGB2, HIST1H1C, EIF2C3, HNRNPC, ILF3, RBM15B, RPL19, MRPL4, DUS2L, TRIP6, GTPBP4, EEF2, DDX54, RPL23A, RRP36, PPAN, PATL1, REXO4, DND1, NOP56, RPS11, RBM15, RPS4X, MRPS15, HNRNPR, PIWIL2, SYNCRIP, SNRPB, RPL32, or FBL.

In some embodiments, the RBP binds downstream of a poly(A) signal of the target RNA, and wherein the RBP activates usage of a given proximal poly(A) signal and/or site of the target RNA. In some embodiments, the RNA binding protein comprises, consists essentially of, or consists of CSTF1, TOB2, PRRC2B, PARN, RBM11, DDX17, GTSF1, DDX5, RBM10, SNRPA, RBM22, CNOT4, RBM14, MBNL1, SUPT5H, ZC3HAV1, RBM4, CNOT2, SAMD4A, CDC40, HNRNPH1, SRP68, EIF4B, CSTF2T, LGALS3, NANOS3, SFPQ, CIR1, PPP1CA, ZMAT3, PRPF4, PRPF4, SCAF8, TRNAU1AP, EDC3, PCBP3, LSM1, PPIA, TPD52L2, RBM5, APOBEC3A, LGALS1, CDC42EP4, DZIP3, HNRNPH2, STAU2, PCBP1, GRB2, NUP35, EIF4H, BTG1, WDR6, THUMPD1, RSRC1, SMNDC1, SYMPK, NONO, MBNL2, MAZ, STAU1, HNRNPF, TFIP11, SMN1, AHNAK, YTHDF1, NGRN, METTL16, SNRPG, SF3A2, STAU2, YWHAE, YWHAG, SFRS2IP, PEG10, HRSP12, EXOSC10, CDC42EP4, LSM7, ZC3H7A, SNRPN, RBM42, AHNAK, GSPT1, SMN1, MECP2, SRSF9, QARS, ASCC3, CUGBP1, ZFC3H1, NMD3, SNRPB, SRSF2, NUSAP1, RBM38, CORO1A, SFRS17A, BTG4, GTPBP10, SNW1, or CPSF6.

In some embodiments, the RBP binds downstream of a poly(A) signal of the target RNA, and wherein the RBP inhibits usage of a given poly(A) signal and/or site of the target RNA. In some embodiments, the RNA binding protein comprises, consists essentially of, or consists of FZD3, PPIG, CSTF3, WDR36, DHX8, SLIRP, CNOT10, SOX21, MRPS11, PURG, ADK, TRAP1, LSM4, NGDN, DYNC1H1, FLYWCH2, NPM1, IGF2BP1, ASS1, GNL2, RBPMS, LSM10, FAM46A, TPT1, RNMTL1, LARP4, CPSF5, LUC7L3, NAA15, RBM3, TPT1, ELAC2, RPGR, PNO1, UTP3, SNRPB2, HIST1H1C, ASCC1, SART3, EIF2C3, EIF4A1, EIF3L, MRPS15, LUC7L, SNRPE, LIN28A, CNOT10, NHP2, PARP12, HADHB, MRPS30, XPO5, RPS15A, NPM1, PURB, MTPAP, MRPL11, COA6, GLE1, NUDT16, GRN, RBM17, DHX57, PHF5A, SMG6, SARNP, SUGP2, TSR1, KRR1, C1D, ESRP1, PSMD4, SRSF10, TRMT10C, ZGPAT, BOLL, SPATS2L, MRPS24, RBM26, RPL6, CLASRP, DDX59, SEC63, EIF2S2, RPL23, DHX37, CPEB1, SNRPF, MRPS31, PNLDC1, MRPS23, RPS3A, TEFM, CCT4, POLR2G, RPS7, CNBP, FUBP1, SRPR, DDX60, NAP1L3, RPL22, EIF3C, DDX27, NOA1, LARP1B, FDPS, USP36, CMSS1, CCNL2, RPP30, SF3B3, MEPCE, SBDS, RPUSD4, U2AF2, RPL21, THOC3, FXR2, RPL6, LSM6, FAM98A, WDR33, DUS2L, THOC5, TRIM39, FAM208A, RRP7A, MRPS35, P4HB, LARP1, MAK16, RBM4, EIF2C2, ARL6IP4, FZD8, FTSJ3, PUS7, IFIT2, EIF2C2, S100A4, DHX58, TRIM56, SRPR, SERBP1, MRPL30, GTF2F1, LSMD1, TFB1M, FKBP3, PHF6, METTL3, ALDH18A1, ZFC3H1, GSPT2, GTPBP4, EXOSC3, PRPF6, RC3H2, NSUN2, DHX35, DCN, ANXA2, DHX40, SAFB2, RPS6, IFIH1, DDX43, NOL12, GFM1, RPS20, PUF60, PIWIL1, RBM8A, LARP6, HSP90AB1, MRPS5, MAP4, MRPL37, PRPF19, XRN2, HFM1, ALDH6A1, FTO, SPATS2, TRMU, USP32, TROVE2, RPS15A, XRCC6, DNAJC17, NOP58, RPL7, RPL10A, DDX1, C16orf88, APOBEC3B, PPAPDC2, HDGF, C1orf131, SUPV3L1, RPS3, TDRD9, RALYL, CPSF2, SUCLG1, DDX20, RPN1, RPP25, PTRH1, EEF1A1, FAM32A, SEC63, CNOT6, ILF3, SRFBP1, TRIP6, NOSIP, FSCN1, NOB1, RPS6, GNL2, SRPK2, EIF3D, RPL15, TRMT6, RPL8, NOP2, CHD2, CIRBP, RBMS1, SERBP1, NOP16, FASTKD2, CPSF4, DDX11, CHTOP, PWP2, SREK1, LSM3, BCCIP, BYSL, SECISBP2, TRMT2A, REPIN1, CIRH1A, MRTO4, RPL19, HADHB, NOL10, SYF2, MFAP1, APOBEC3C, PRPF38B, RPS2, NSA2, CPSF3L, SNRNP35, THOC6, DDX11, RPL13A, DDX31, NPM1, BST2, PRPF40A, PES1, UCHL5, MKI67IP, FIP1L1, RPL8, MRPL42, ATP5C1, KRR1, RPL15, RPLP0, PUF60, ZRANB2, AIMP1, DDX54, MRPL4, SF3A1, POLR3E, RBM33, THRAP3, GNL3L, RPL4, DDX55, DHX33, RPL3, RPS8, DDX27, ERAL1, RRS1, PRR3, RNPC3, DDX50, RPL32, DDX39A, DDX39A, RPL5, RBFOX2, PPIG, RPS11, THOC4, RRP36, RPS4X, CCNL1, GLTSCR2, RBM46, RBM15, DHX16, CPSF4L, CPSF4, BMS1, BUD13, PRPF3, MRPL39, DKC1, KIAA0020, CLK3, RTN4, RPL23A, DDX18, RPL14, EDF1, CELF4, DDX55, SAMSN1, ZRSR2, DIEXF, RPL15, DHX30, SRSF4, SRP14, ZCRB1, LARP7, CPSF7, ELAVL2, FRG1, RPS24, DAZ3, NAT10, SRSF7, MKI67IP, DND1, DDX47, PA2G4, SRSF8, RPS4X, MRPL3, RSL1D1, PABPC5, NOC2L, APOBEC3D, UTP11L, G3BP2, ACAA2, DDX28, YTHDC1, RBM19, NOP9, SNIP1, RBM28, C1orf35, SFRS10, SFRS17A, CCDC59, RPL27, EIF2C1, NOL7, RBM34, LYAR, RPL28, KRR1, RBMX, PUF60, REXO4, PPAN, CCDC137, ZRSR1, SRSF12, ZRSR2, U2AF1, SRSF5, ADAD2, RBMX2, SRSF10, UTP23, SRSF6, ZC3H18, GPATCH4, HNRNPD, ABT1, TOP3B, HNRNPA1, RPL35, ELAVL4, RBM47, RRP8, RBM15B, PPIL4, ANXA2, RTCA, NOP56, GTF2E2, CPEB4, ZCCHC17, HNRNPR, PIWIL2, EEF2, RNPS1, RDBP, DDX49, RPL35, DDX52, FBL, ZFP36L1, DDX23, NOLC1, RBMX2, HNRNPCL1, TRA2A, HNRNPC, or HERC5.

### Conjugation of RBP and Gene-Targeting Agent

In some embodiments, the methods described herein can include assembling an RNA regulation unit, wherein the RNA regulation unit comprises, consists essentially of, or consists of an RNA binding protein (RBP) and a gene-targeting agent. In some embodiments, assembling of the RNA regulation unit can be performed outside of a host cell. In some embodiments, the assembling can include plasmid construction.

Further aspects relate to a conjugate comprises, consists essentially of, or consists of RBP operably linked to a gene-targeting agent, wherein the gene-targeting agent. In some embodiments, the RBP and the gene-targeting agent can be conjugated to create one functional RNA regulation unit. In some embodiments, the RBP and the gene-targeting agent are operably linked through a peptide bond. In some embodiments, the polypeptide further comprises, consists essentially of, or consists of one or more linkers. In some embodiments, the RBP and the gene-targeting agent are operably linked through non-covalent interactions. In some embodiments, the RBP is covalently linked to a first dimerization domain and the gene-targeting agent is covalently linked to a second dimerization domain and wherein the first and second dimerization domain are capable of dimerizing to form a non-covalent or covalent linkage. In some embodiments, the conjugate comprises, consists essentially of, or consists of one or more nuclear localization signals (NLS)s.

In certain embodiments, oligomeric compounds are modified by covalent attachment of one or more conjugate groups. Conjugate groups are routinely used in the chemical arts and are linked directly or via an optional linking moiety or linking group to a parent compound such as an oligomeric compound. An exemplary list of conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

Certain conjugate groups include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethyl-ammonium-1,2-di-O-hexadecyl -rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Linking groups or bifunctional linking moieties such as those known in the art can be used as provided herein. Linking groups are useful for attachment of chemical functional groups, conjugate groups, reporter groups and other groups to selective sites such as for example an oligomeric compound. In general, a bifunctional linking moiety comprises, consists essentially of, or consists of a hydrocarbyl moiety having two functional groups. One of the functional groups is selected to bind to a parent molecule or compound of interest and the other is selected to bind essentially any selected group such as chemical functional group or a conjugate group. In some embodiments, the linker comprises, consists essentially of, or consists of a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like. Some nonlimiting examples of bifunctional linking moieties include 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other linking groups include, but are not limited to, substituted C1-C10 alkyl, substituted or unsubstituted C2-C10 alkenyl or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In some embodiments, RNA regulation unit is less than, more than, or are at most or at least 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25, 20, 15, 10, or 5 kDa (or any derivable range therein).

### Delivery

In some embodiments, the methods described herein can include delivering the RNA regulation unit into a cell. In some embodiments, the delivering step comprises, consists essentially of, or consists of a virus-based delivery. In some embodiments, the virus-based delivery comprises, consists essentially of, or consists of adeno-associated virus or lentivirus.

A “delivery vehicle” is defined as any molecule that can carry inserted polynucleotides into a host cell. Examples of delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide disclosed herein can be delivered to a cell or tissue using a delivery vehicle. “Gene delivery,” “gene transfer,” “transducing,” and the like as used herein, are terms referring to the introduction of an exogenous polynucleotide into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of “vectors” are known to be capable of mediating transfer of genes to mammalian cells, as is known in the art and described herein.

A “viral vector” is a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. Further details as to modern methods of vectors for use in gene transfer may be found in, for example, Kotterman et al. (2015) Viral Vectors for Gene Therapy: Translational and Clinical Outlook Annual Review of Biomedical Engineering 17.

In some embodiments, the term “adeno-associated virus” or “AAV” as used herein refers to a member of the class of viruses associated with this name and belonging to the genus dependoparvovirus, family Parvoviridae. Multiple serotypes of this virus are known to be suitable for gene and/or RNA delivery; all known serotypes can infect cells from various tissue types. At least 11, sequentially numbered, are disclosed in the prior art. Non-limiting exemplary serotypes useful in the methods disclosed herein include any of the 11 serotypes, e.g., AAV2 and AAV8. In some embodiments, “AAV” refers to any one of the serotype AAV1, AAV2, AAV4, AAV5, AAV6, AAV7, AAV8, AAV9, AAV10, AAV11, AAV12, AAV13, AAV PHP.B, or AAV rh74.

Lentiviral vectors of this disclosure are based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to the disclosure may be based on a genetically or otherwise (e.g. by specific choice of packaging cell system) altered version of a particular retrovirus.

That the vector particle according to the disclosure is “based on” a particular retrovirus means that the vector is derived from that particular retrovirus. The genome of the vector particle comprises components from that retrovirus as a backbone. The vector particle contains essential vector components compatible with the genome, such as an RNA genome, including reverse transcription and integration systems. Usually these will include gag and pol proteins derived from the particular retrovirus. Thus, the majority of the structural components of the vector particle will normally be derived from that retrovirus, although they may have been altered genetically or otherwise so as to provide desired useful properties. However, certain structural components and in particular the env proteins, may originate from a different virus. The vector host range and cell types infected or transduced can be altered by using different env genes in the vector particle production system to give the vector particle a different specificity.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene.

Delivery vehicles also include DNA/liposome complexes, micelles and targeted viral protein-DNA complexes. Liposomes that also comprise a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

In some embodiments, delivering includes lipofection/lipid transfection/liposome-based transfection. One embodiment of the disclosure contemplates liposomes capable of attaching and releasing nucleic acid conjugates, polypeptides, and/or fusion proteins as described herein. Liposomes are microscopic spherical lipid bilayers surrounding an aqueous core that are made from amphiphilic molecules such as phospholipids. For example, a liposome may trap a nucleic acid between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phospholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present disclosure contemplates an ultra high-shear technology to refine liposome production, resulting in stable, unilamellar (single layer) liposomes having specifically designed structural characteristics. These unique properties of liposomes allow the simultaneous storage of normally immiscible compounds and the capability of their controlled release.

The following examples are provided to illustrate, and not limit the disclosure.

### EXAMPLES

### Example 1—Determine Efficiency and Effect of an RNA Binding Protein

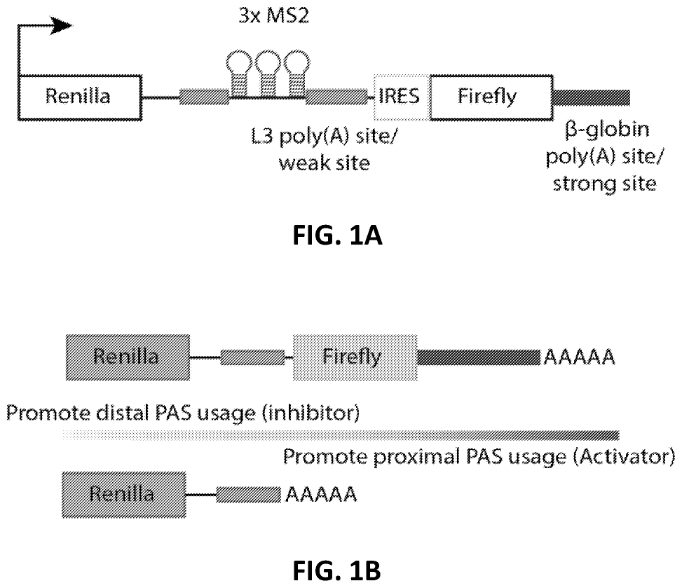

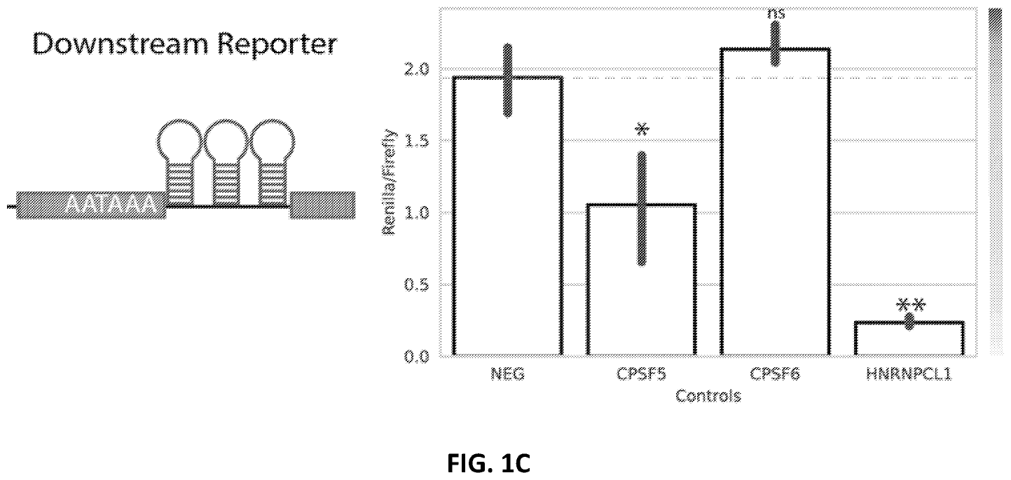

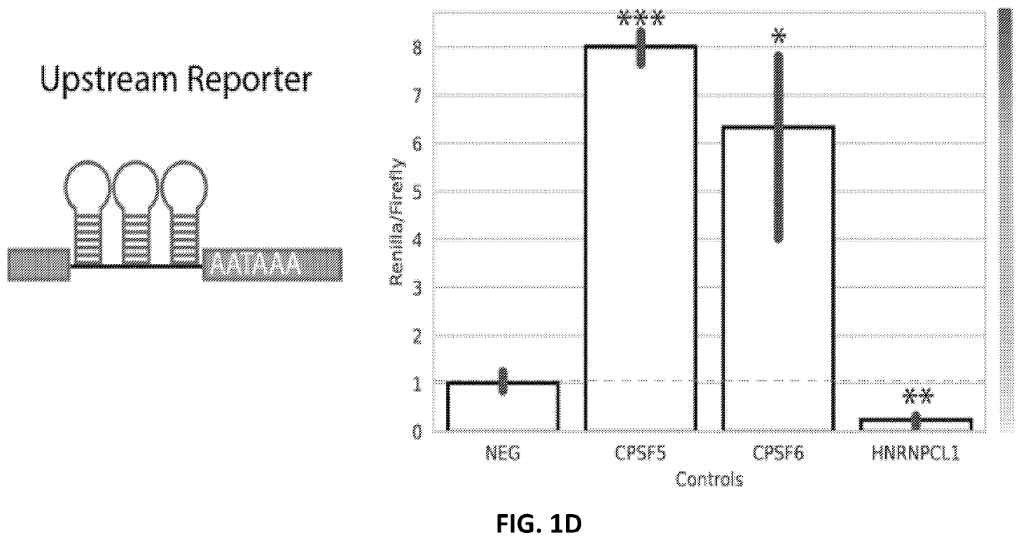

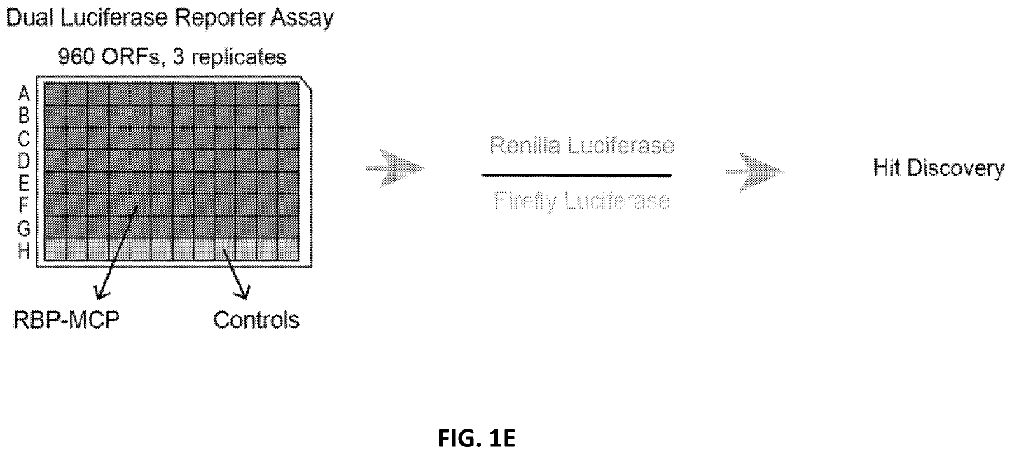

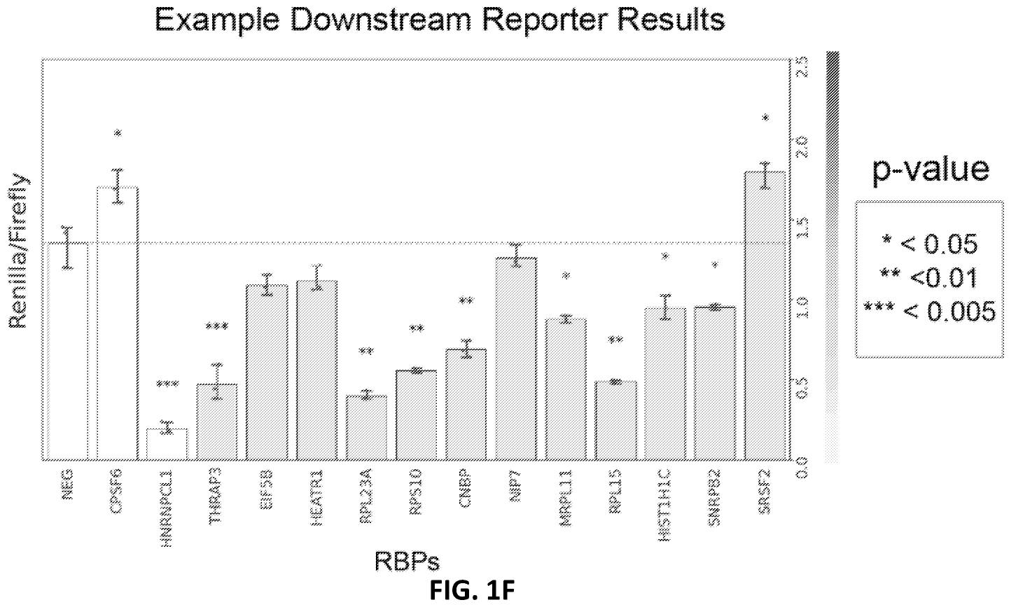

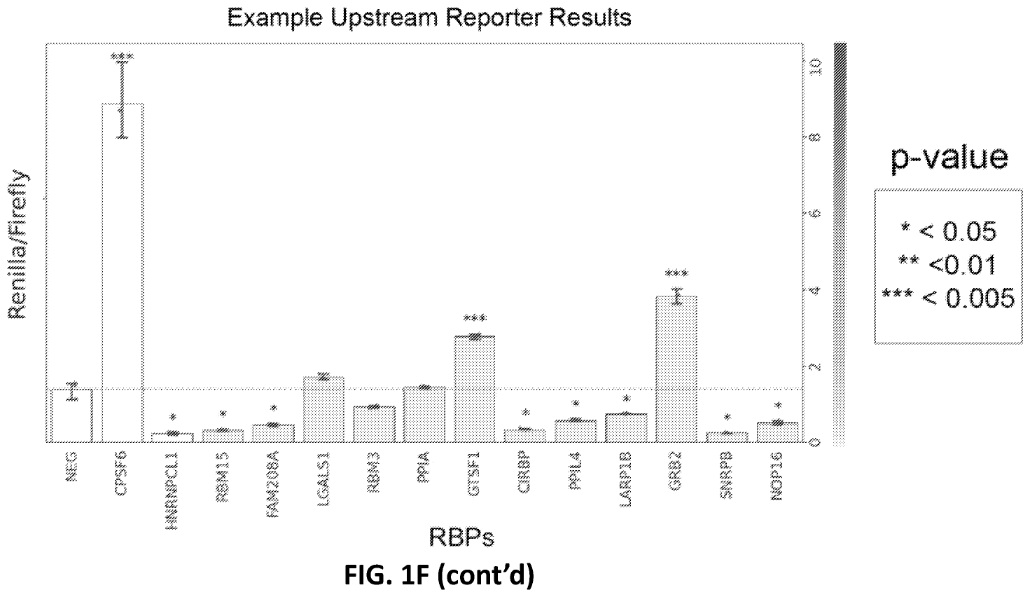

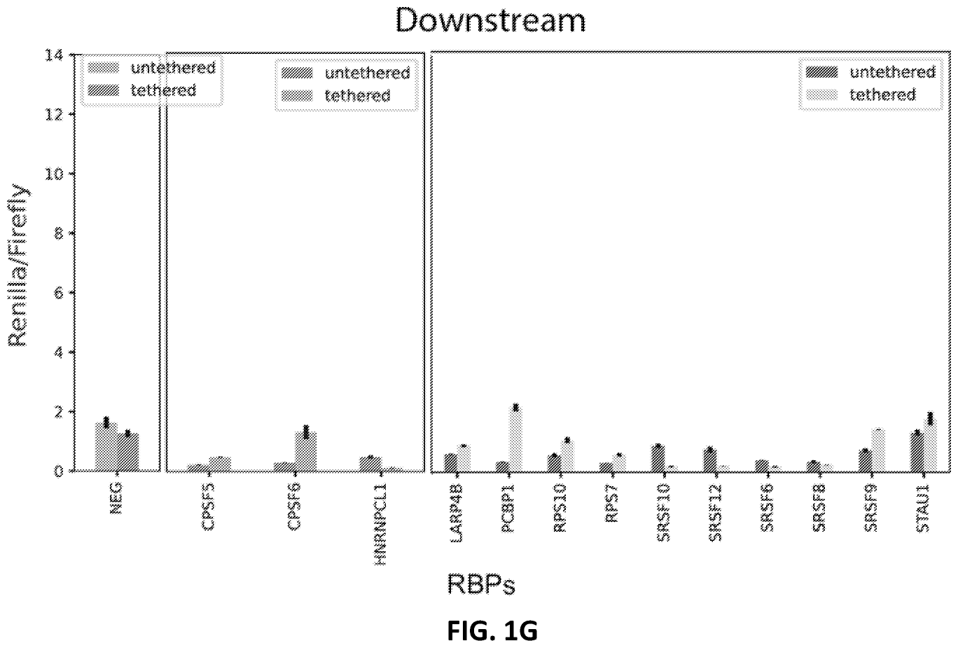

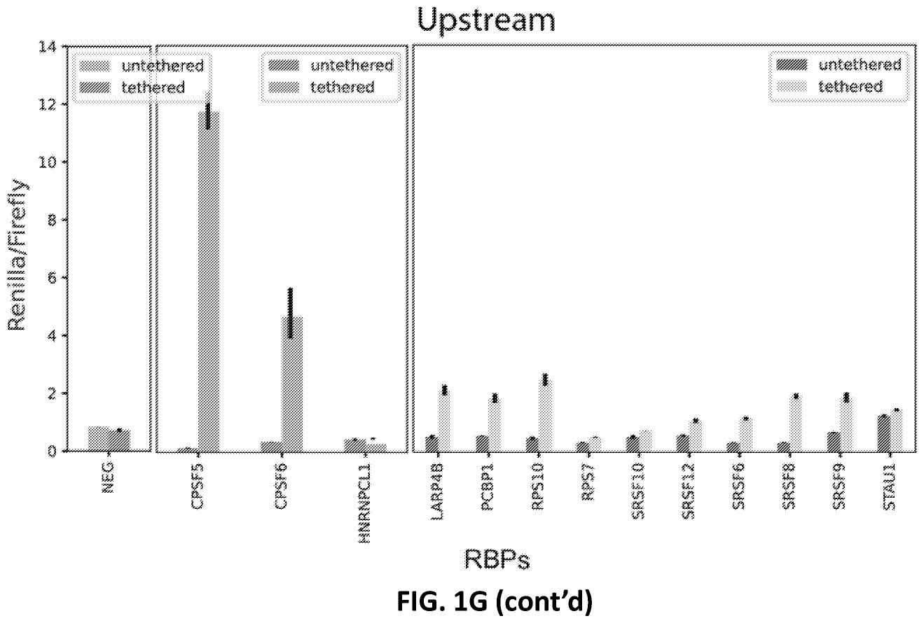

Using a tethered function assay and dual-luciferase reporter system, over 600 RBPs fused to the bacteriophage MS2 coat protein (MCP) were screened to determine the efficiency and effect of an RBP binding upstream or downstream of a proximal poly(A) site on poly(A) site selection and usage (FIGS. 1A-1B). To measure the efficiency and to categorize the effect on regulating APA as promoting the proximal poly(A) site or the distal poly(A) site, the ratio of Renilla/Firefly was calculated which represents the ratio of proximal poly(A) site usage to distal poly(A) site usage (FIG. 1E-1F). To ensure the method was properly determining effects of RBP binding, the effects of a negative flag (no RBP binding) and control RBPs having known effects were measured with each batch of RBPs tested (FIGS. 1C-1D). Because the method employs a tethering assay, the readouts of tethered and untethered versions of a select few RBPs were compared to make sure effects seen were due to tethering and not overexpression of the RBP (FIG. 1G).

### Example 2—Identify RBP Candidates That Show Significant Effects Promoting/Inhibiting Poly(A) Site Usage

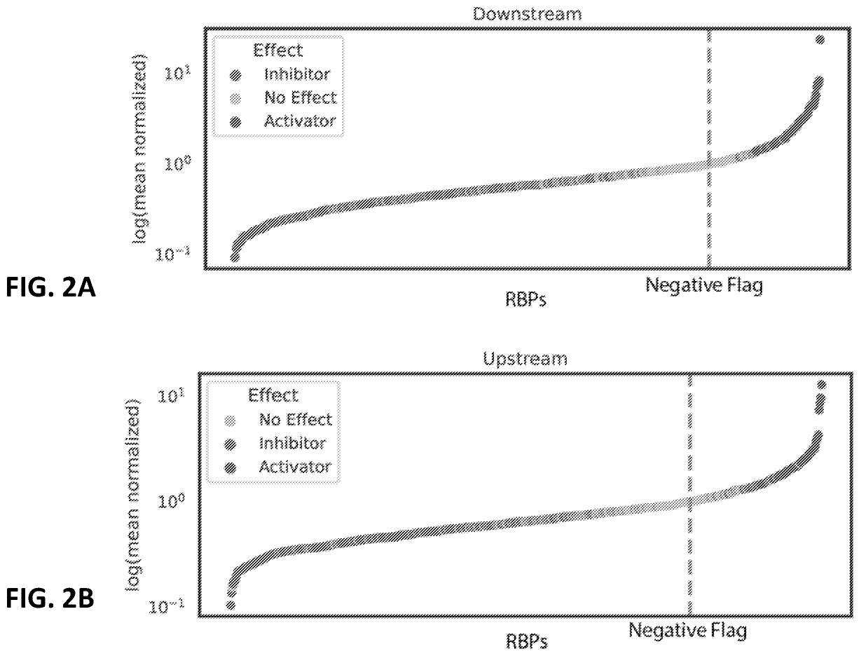

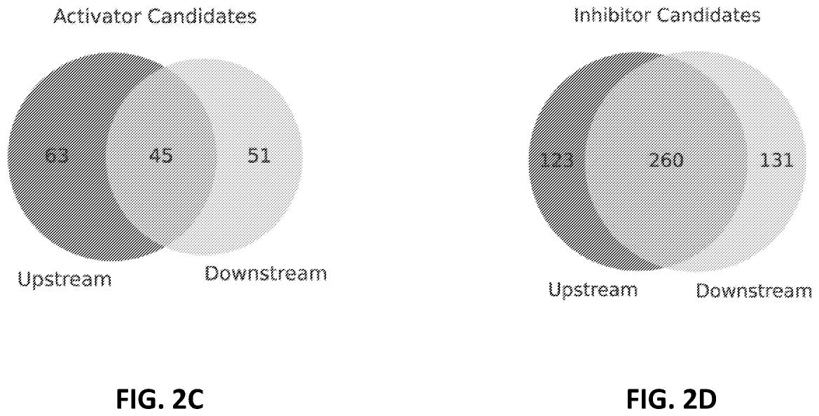

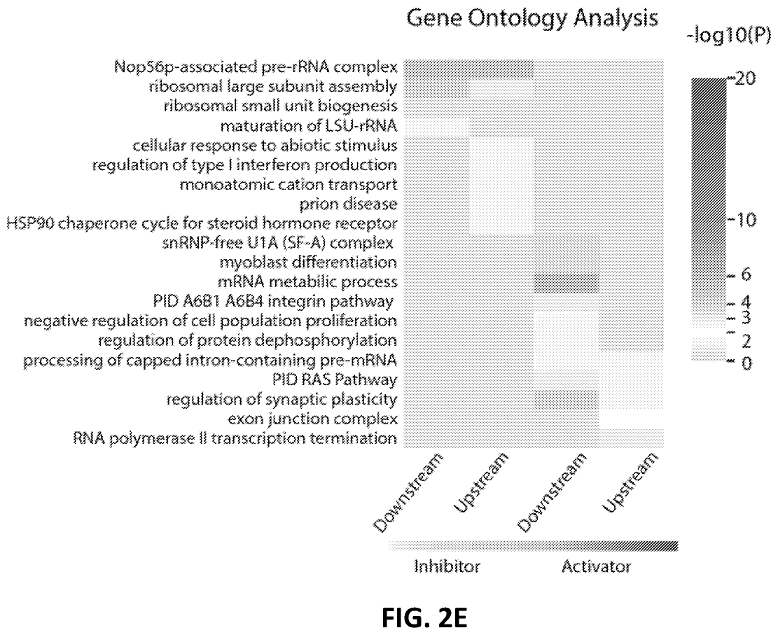

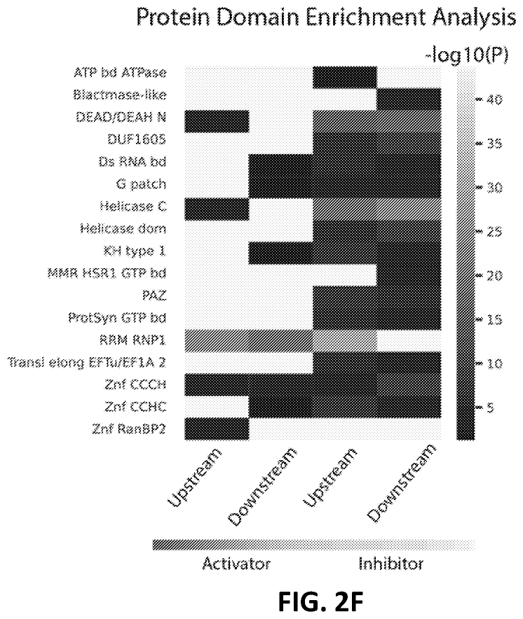

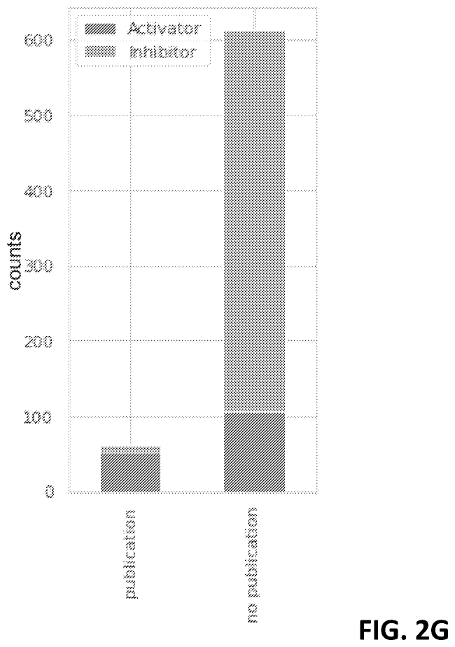

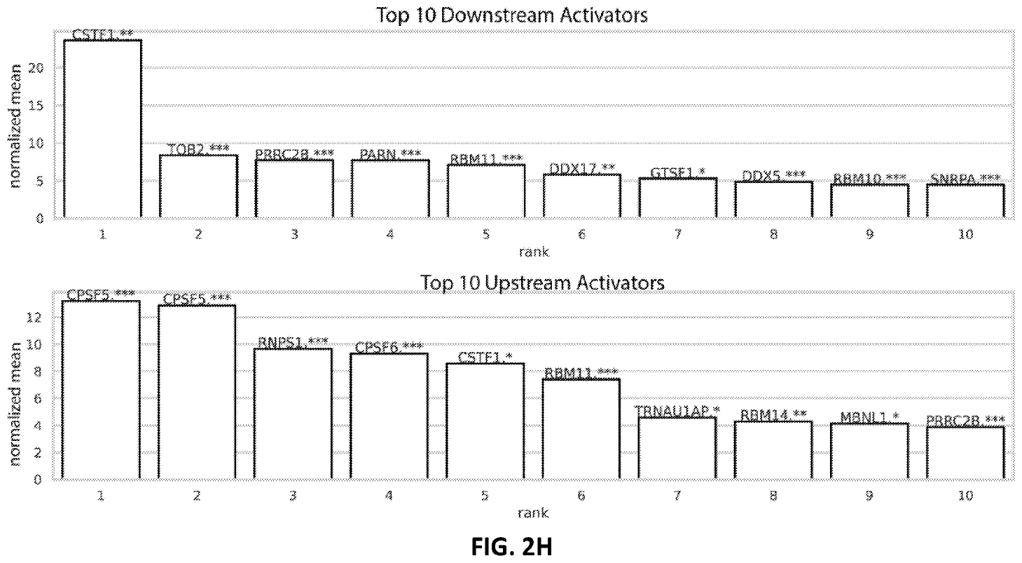

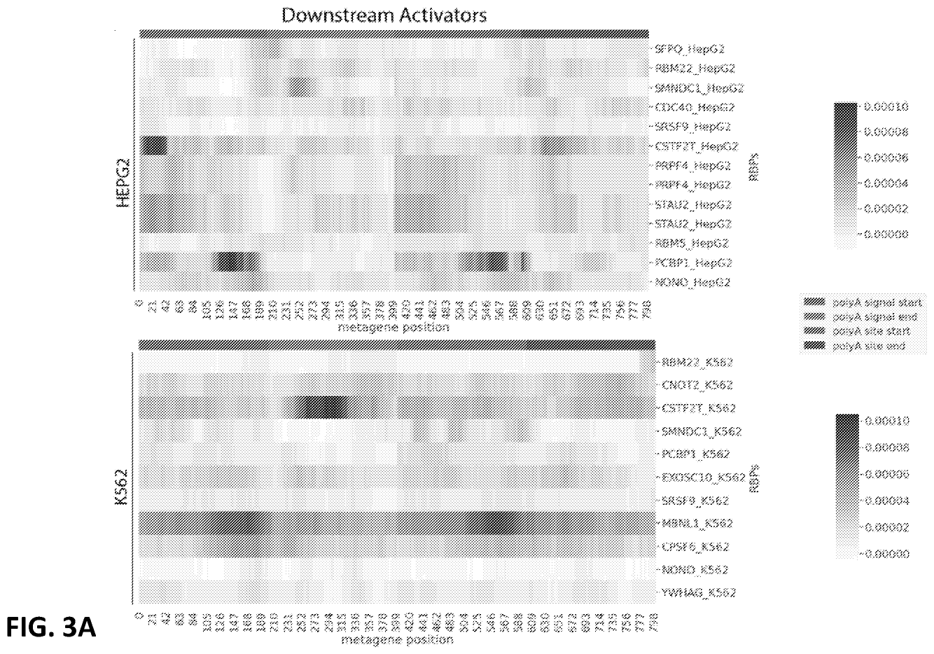

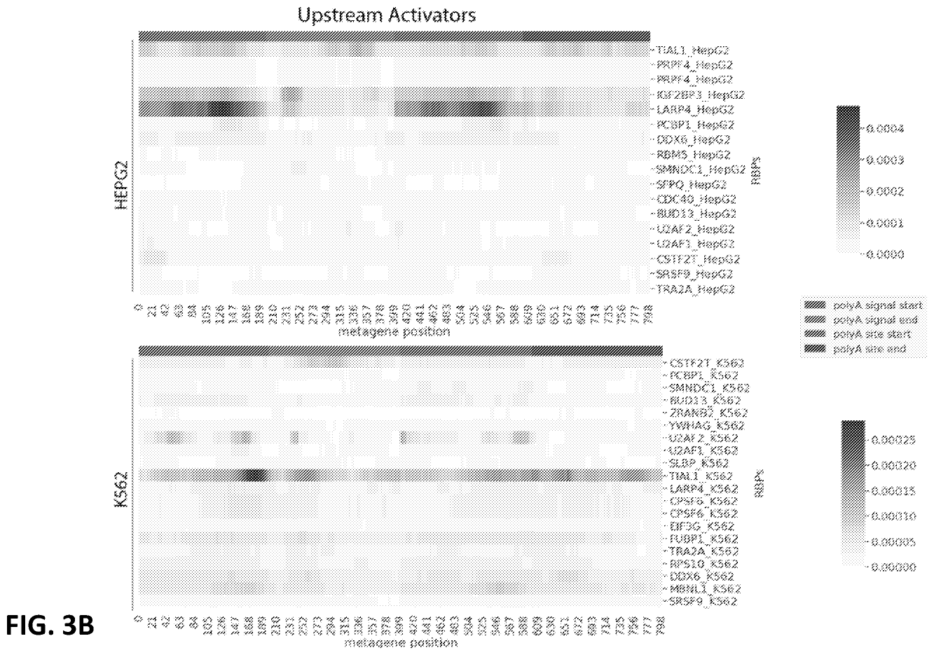

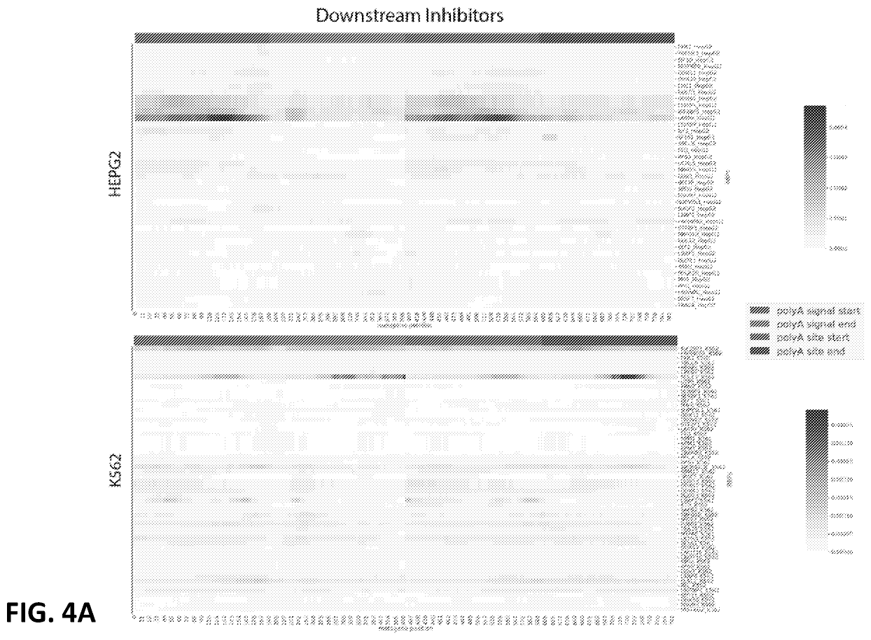

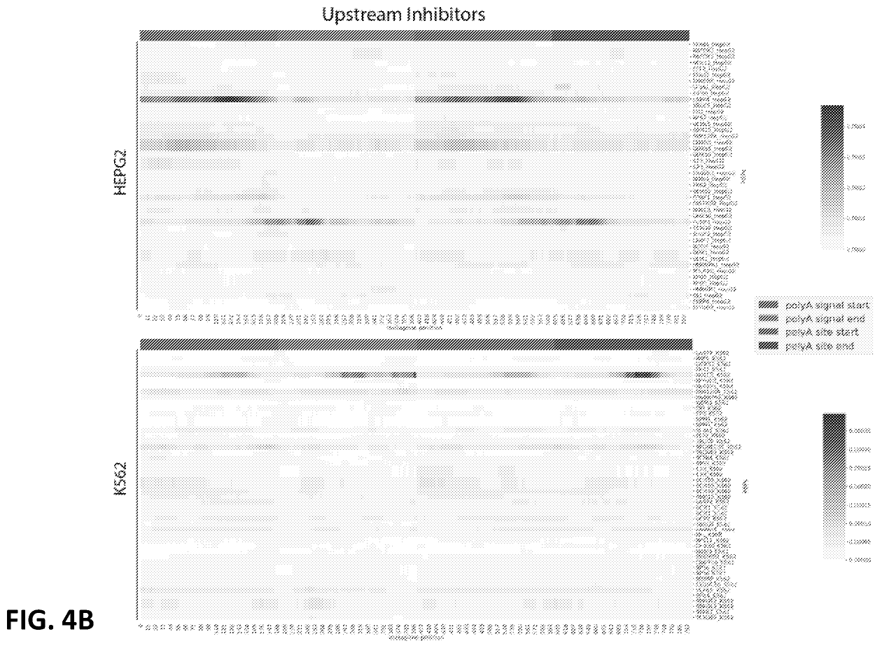

Overall, RBP candidates were identified that show significant effects promoting proximal poly(A) sites or promoting distal poly(A) site usage of various levels of efficiency following binding upstream or downstream of proximal poly (A) sites (FIGS. 2A-2D). The candidates that promote proximal poly(A) site usage are associated with distinct Gene Ontology classifications from those that promote distal poly(A) site usage (FIG. 2E) and many of these candidates have not previously been associated specifically with APA (FIG. 2G). Based on available ENCODE data, it also appeared that many of these RBP candidates do not have a role in APA normally in mammalian cells (FIGS. 3A-3B, 4A-4B). Protein domain enrichment analysis was performed to determine enrichment for protein domains in significant activating or inhibiting effects on proximal poly (A) site selection (FIG. 2F). Furthermore, the list of candidate RBPs were ranked by measuring efficiency and categorizing by binding location-specific effects on poly(A) site usage (FIG. 2H).

### Example 3—RBP Candidates to be Used With Existing Systems to Regulate APA

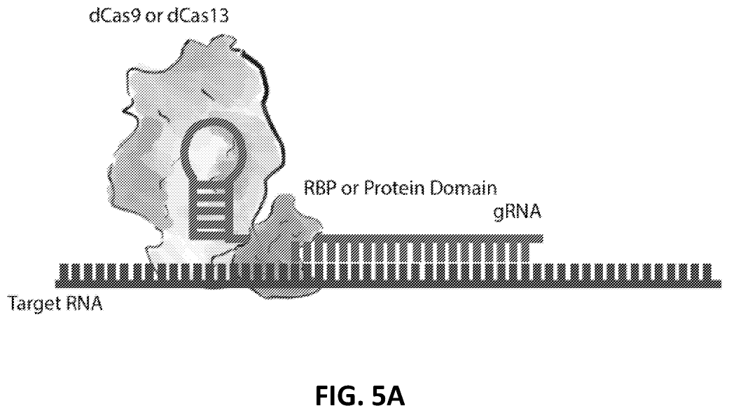

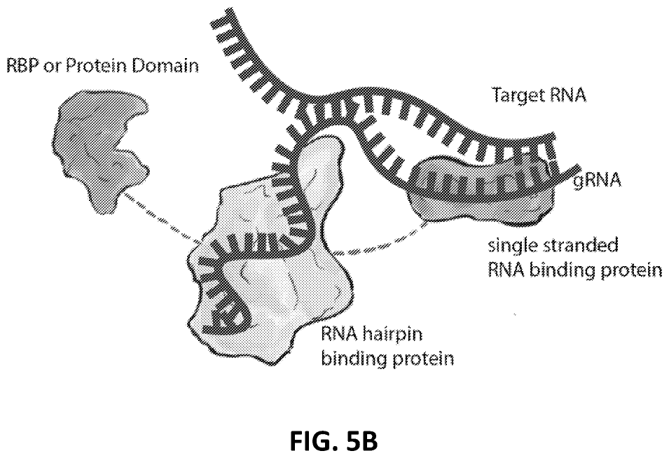

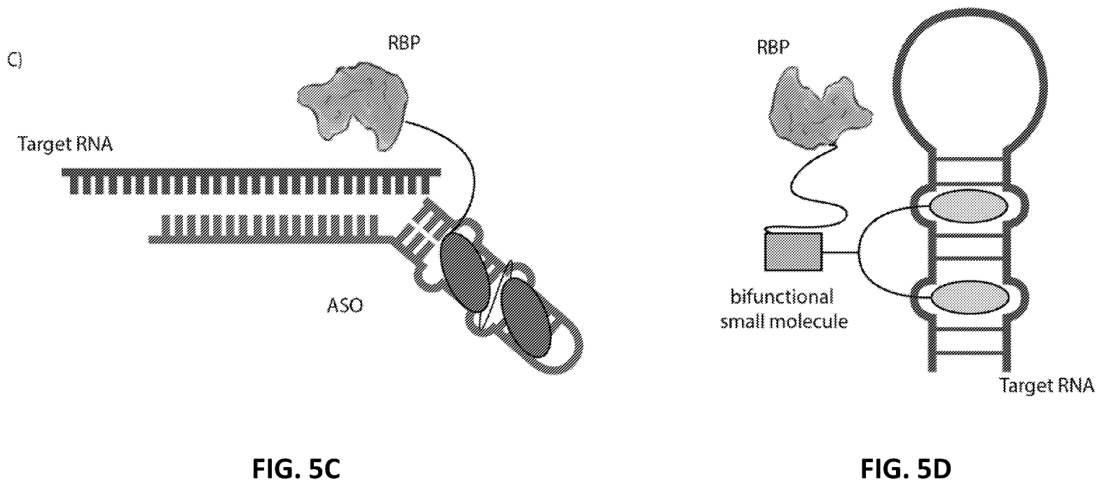

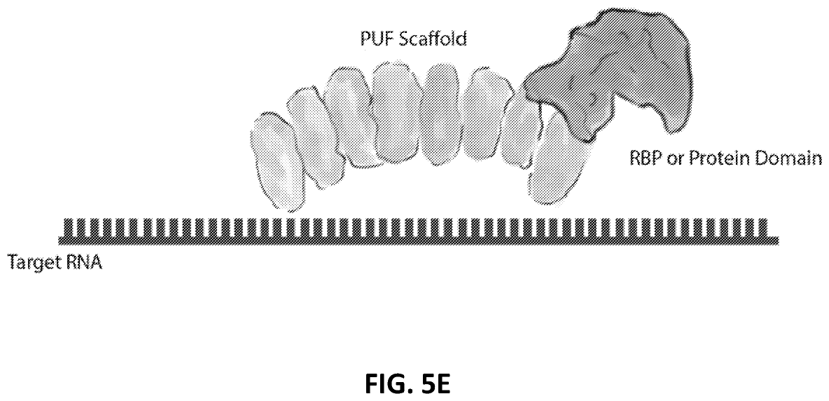

Existing systems that can be coupled with the list of RBP candidates for regulating APA of target RNA transcripts were identified (FIGS. 5A-5E). The RBP candidates list can be used to select the appropriate RBP based on the location of binding and effect on APA required. Though many of these RBPs have not previously been associated with APA (FIG. 2G), they were shown to be effective regulators of APA. This was further supported by data from the ENCODE consortium. For RBPs with data available in the ENCODE database, binding profiles in the poly(A) site regions indicated that some RBPs bind near them, implying they have a role in the process of APA in mammalian cells, and others do not (FIGS. 3A-3B, 4A-4B).

By coupling this list of RBPs with existing molecular tools in order to modulate APA, regulation would occur at the RNA level. In addition, it would allow for enhancing usage of proximal or distal poly(A) sites.

### Example 4—RBPs for Regulating Alternative Polyadenylation (APA) of a Target RNA in a Cell

An RBP is selected from the RBPs identified herein for the properties the RBP is identified has having (e.g., binding upstream, binding downstream of a polyadenylation signal, inhibiting alternative polyadenylation or activates alternative polyadenylation). The selected RBP is operably linked to an inactive Cas9 to form an RNA regulation unit. Using this modality, a guide RNA (gRNA) is designed such that it guides the system to the mRNA target of interest (FIG. 5A). The RNA regulation unit is then delivered to a cell. Monitoring of alternative polyadenylation of a target RNA in a cell reveals the RNA regulation unit comprising an inactive Cas with the gRNA and selected RBP, modulates APA in the expected direction.

### Example 5—RBPs for Increasing Stability of a Target RNA in a Cell

An RBP is selected from the RBPs identified herein for the properties the RBP is identified has having (e.g., binding upstream, binding downstream of a polyadenylation signal, inhibiting alternative polyadenylation or activates alternative polyadenylation). The selected RBP is operably linked to create a CIRTS system. The CIRTS system is a programmable RNA-targeting platform that has the ability to deliver effectors to target RNA transcripts using a complex made up of a gRNA, single-stranded RNA binding protein, and an RBP candidate (FIG. 5B), wherein the gRNA is designed to guide the system to the mRNA target of interest. The RNA regulation unit is then delivered to a cell. Monitoring of stability of a target RNA in a cell reveals the RNA regulation unit comprising the CIRTS system and selected RBP, increases stability of the target RNA.

### Example 6—RBPs for Preventing Degradation of a Target RNA in a Cell

An RBP is selected from the RBPs identified herein for the properties the RBP is identified has having (e.g., binding upstream, binding downstream of a polyadenylation signal, inhibiting alternative polyadenylation or activates alternative polyadenylation). ASOs are small, single-stranded nucleic acids that target RNA transcripts. An ASO is engineered such that it recruits a selected RNA binding protein candidate, and targets the target RNA transcript (FIG. 5C). The RNA regulation unit is then delivered to a cell. Monitoring of degradation of a target RNA in a cell reveals the RNA regulation unit comprising the ASO system and selected RBP, decreases degradation of the target RNA.

### Example 7—RBPs for Modifying Localization of a Target RNA in a Cell

An RBP is selected from the RBPs identified herein for the properties the RBP is identified has having (e.g., binding upstream, binding downstream of a polyadenylation signal, inhibiting alternative polyadenylation or activates alternative polyadenylation). The selected RBP is operably to a bifunctional small molecule. Small molecules are organic compounds that have low molecular weight. The small molecule is engineered to recruit a selected RBP to a target RNA transcript (FIG. 5D). The RNA regulation unit is then delivered to a cell. Monitoring of localization of a target RNA in a cell reveals the RNA regulation unit comprising the bifunctional small molecule system and selected RBP, decreases degradation of the target RNA.

### Example 8—RBPs for Increasing Synthesis of a Protein Encoded by a Target RNA in a Cell

An RBP is selected from the RBPs identified herein for the properties the RBP is identified has having (e.g., binding upstream, binding downstream of a polyadenylation signal, inhibiting alternative polyadenylation or activates alternative polyadenylation). The selected RBP is operably to a PUF protein. PUF proteins have the ability to bind any RNA of interest and by using this modality, a candidate RBP is fused to a PUF scaffold to increase synthesis of a protein encoded by a target RNA (FIG. 5E). The RNA regulation unit is then delivered to a cell. Monitoring of target protein production in a cell reveals the RNA regulation unit comprising the PUF protein and selected RBP, increases synthesis of a protein encoded by the target RNA.

### Equivalents

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method of regulating alternative polyadenylation (APA) of a target RNA in a cell, the method comprising:
(a) Assembling an RNA regulation unit, wherein the RNA regulation unit comprises an RNA binding protein (RBP) and a gene-targeting agent, wherein the RNA binding protein binds proximal to a poly(A) signal and/or site;
(b) delivering the RNA regulation unit into the cell, wherein the RNA regulation unit regulates alternative polyadenylation (APA) of the target RNA in the cell; and
(c) detecting a change in the target RNA translation.

2. A method of increasing stability of a target RNA in a cell, the method comprising:
(a) assembling a RNA regulation unit, wherein the RNA regulation unit comprises an RNA binding protein (RBP) and a gene-targeting agent, wherein the RNA binding protein binds proximal to a poly(A) signal and/or site;
(b) delivering the RNA regulation unit into the cell, wherein the RNA regulation unit increases stability of the target RNA in the cell; and
(c) detecting a change in the target RNA translation.

3. A method of preventing degradation of a target RNA in a cell, the method comprising:
(a) assembling a RNA regulation unit, wherein the RNA regulation unit comprises an RNA binding protein (RBP) and a gene-targeting agent, wherein the RNA binding protein binds proximal to a poly(A) signal and/or site;
(b) delivering the RNA regulation unit into the cell, wherein the RNA regulation unit prevents degradation of the target RNA in the cell; and
(c) detecting a change in the target RNA translation.

4. A method of modifying localization of a target RNA in a cell, the method comprising:
(a) assembling a RNA regulation unit, wherein the RNA regulation unit comprises an RNA binding protein (RBP) and a gene-targeting agent, wherein the RNA binding protein binds proximal to a poly(A) signal and/or site;
(b) delivering the RNA regulation unit into the cell, wherein the RNA regulation unit modifies localization of the target RNA in the cell; and
(c) detecting a change in the target RNA translation.

5. A method of increasing synthesis of a protein encoded by a target RNA in a cell, the method comprising:
(a) assembling a RNA regulation unit, wherein the RNA regulation unit comprises an RNA binding protein (RBP) and a gene-targeting agent, wherein the RNA binding protein binds proximal to a poly(A) signal and/or site;
(b) delivering the RNA regulation unit into the cell, wherein the RNA regulation unit increases synthesis of the protein encoded by the target RNA in the cell; and
(c) detecting a change in the target RNA translation.

6. The method of claim 1, wherein the RBP binds to a plurality of poly(A) signals and/or sites.

7. The method claim 1, wherein the RNA binding protein binds to the poly(A) site.

8. The method of claim 1, wherein the gene-targeting agent is selected from CRISPR components, a Cas RNA targeting system or a non-Cas RNA-targeting system, optionally wherein the Cas RNA targeting system is an inactive Cas13 (dCas13) or an inactive RNA-targeting Cas9 (dCas9) and optionally wherein the non-Cas RNA-targeting system comprises a CRISPR-cas inspired RNA targeting system (CIRTS).

9-13. (canceled)

14. The method of claim 1, wherein the gene-targeting agent comprises small molecules, or engineered protein domains.

15. The method of claim 14, wherein the engineered protein domain comprises a PUF domain.

16. The method of claim 1, wherein the gene-targeting agent comprises shRNAs, siRNAs, antisense oligonucleotides (ASOs), or microRNA mimics.

17. The method of claim 1, wherein the delivering step (b) comprises lipofection or a virus-based delivery.

18. (canceled)

19. The method of claim 17, wherein the virus-based delivery comprises adeno-associated virus or lentivirus.

20. The method of claim 1, wherein the target RNA is an endogenous mRNA or a non-coding RNA.

21. (canceled)

22. The method of claim 1, wherein the RBP binds upstream of a poly(A) signal and/or site of the target RNA, and wherein the RBP activates poly(A) site selection of the target RNA, optionally wherein the RBP is CPSF5, RNPS1, CPSF6, CSTF1, RBM11, TRNAU1AP, RBM14, MBNL1, PRRC2B, EIF4B. LGALS1, LUC7L, APOBEC3A, FUBP1, CDC40, UBE2I, SRP68, NGRN, ZRANB2, GRB2, RBM5, ZC3H18, PRPF40A, TIAL1, RBM10, ZC3HAV1, RPS10, YTHDF1, EIF4A3, IGF2BP3, SAMD4A, PNN, CLK2, PRPF4, RPS28, EIF4H, RY1, LARP4B, EIF3G, FLYWCH2, CIR1, WDR6, SMNDC1, SLBP, GTSF1, U2AF2, PRPF4, RBBP6, SRSF8, MBNL2, SRSF9, PCBP1, SBDS, PPIA, RPS19BP1, ISY1, CPSF6, DNAJC17, TOB2, RBM38, RPS21, LGALS3, CPSF7, DDX6, TRA2A, BUD13, SNRPA, STXBP1, RPL30, LSM1, GNB2L1, SARNP, HFM1, CNOT3, METTL16, LSM5, CCDC75, CHAF1B, STAU1, AHNAK, RBM8A, MAZ, NOSIP, PARN, SPATS2L, SNRNP40, YWHAG, LSM10, SNRNP70, PHF5A, RBFOX1, TPD52L2, RBM18, BCDIN3D, U2AF1, CSTF2T, SFPQ, SUPT5H, LARP4, EDC3, ASCC1, MKI67IP, EIF4A1, PDIA3, SSRP1, HNRNPCL1, PAPOLG, EDF1, RBM12B, or DCD.

23. (canceled)

24. The method of claim 1, wherein the RBP binds upstream of a poly(A) signal and/or site of the target RNA, wherein the RBP inhibits poly(A) site selection of the target RNA, and optionally wherein the RBP the RBP is RBPMS, RPL23, DHX16, NDUFV3, LUC7L3, RPL31, SRSF3, RPP21, SUMO1, FKBP4, FASN, DDX39A, EZR, RBFOX2, ZCRB1, RAN, AHNAK, SRSF11, SERBP1, PIWIL4, FUBP3, ERCC3, EIF2C2, DNAJC2, RBM26, NUDT16L1, SLIRP APOBEC3C, LSMD1, SRBD1, RPS3A, SSBP1, WDR3, RPL36, SUGP1, STIP1, SLC3A2, IFIT2, RPL23, ELMOD3, CPSF1, APOBEC3G, GLRX3, XPO5, COA6, CPSF4, HSPD1, PTRF, DCP2, XPO5, RPS15A, DDX20, GRN, PHF6, SUGP2, RBMS1, SF1, RBM33, SRPR, NAA15, DCN, DIS3, SF3B2, TRAP1, MTPAP, FTO, C1orf131, RPL3, IFI16, USP36, RBM25, EXOSC3, NPM1, MRPS5, CCDC9, SEC61B, ZCCHC7, FAM120A, DNTTIP2, NOLC1, EIF2S2, SAFB2, SNRPE, ILF2, RDBP, THOC5, DDX1, MRPS11, PUS7L, RPL6, NPM1, SF3B4, CLP1, TRIM56, CALR, DHX58, RPS6, ARL6IP4, PRPF38B, APOBEC3B, REPIN1, RBM45, MEPCE, CNOT6, EIF2C2, DMGDH, NOA1, ASS1, RPS3, EIF5B, RBM17, EXOSC10, NSA2, DCP1A, SART3, APEH, TEFM, BTG4, BCCIP, PNLDC1, EIF3H, HSP90AA1, TSR1, CLASRP, CPNE3, PSMD4, FAM98A, PUF60, G3BP1, PRPF19, RPS6, RBFOX2, CPEB1, SEC63, RPL15, NHP2, ZCCHC11, HSPA9, RTN4, NONO, ZCCHC7, EIF2AK2, RPL21, DHX40, ELAVL4, TUFM, RPL5, DHX8, SRFBP1, RPS20, SNRPG, PWP2, THOC3, SUCLG1, PDIA4, ZMAT3, ZYX, DHX36, FAM98A, DDX19B, ANXA2, POLK, FTSJ3, LARP1B, ANXA2, C1D, ACAA2, PSMC1, DDX18, PPP1CB, PSPC1, LARP4, CNOT10, FANCM, MRPL11, DDX27, DDX11, SEC63, HEATR1, NOP2, RPS7, DYNC1LI1, SAMSN1, DHX35, EIF3L, PABPN1L, RRS1, HADHB, NUDT16, SLC7A9, CPSF4, MRPL37, ERAL1, RPL8, RNMTL1, SRP14, PPAPDC2, NPM3, CORO1A, STAU2, RPL28, CELF4, RPUSD4, YTHDC1, DIMT1, HADHB, TBRG4, DDX55, FASTK, ALDH6A1, CPSF2, SF3B3, SNRPB2, PUS7, RPL35, DDX53, MDH2, FXR2, ILF3, METTL3, TRMT2A, HSPA8, SF3A1, TPT1, FDPS, CLK3, RRP8, RPS8, GPANK1, LARP6, TDRD9, TARDBP, ATP5C1, MRPS11, PCBP4, RBM26, UTP11L, RPL4, CCDC137, PINX1, RNMTL1, FZD8, NAP1L3, POLR2G, DDX31, RPS2, DDX41, SRPR, RBM28, RPN1, RPS24, NXF1, G3BP2, U2AF1, RTCA, RBM46, BYSL, DYNC1H1, PRR3, POLR3E, RBM6, PUF60, RTN4, GANAB, TSR1, TRUB2, DDX28, TERT, DIEXF, NOP58, APOBEC3D, IFIH1, QKI, KIAA1324, EEF1A1, RALY, CNOT7, RPS4X, DDX39A, FASTKD2, NOL10, DDX11, PES1, EFTUD2, PPIL4, RSL1D1, SNRPN, KRR1, MRPL32, ELAVL2, NANOS3, CIRBP, CCDC59, DDX49, ZFC3H1, RPL15, ALDH18A1, HERC5, MRM1, CNBP, RPLP0, KRT18, TOP3B, FSCN1, SLC25A5, SURF6, CPSF4L, RPL14, MRPL39, THOC6, PKM, RPL3, NOL7, DDX43, GSPT2, NOP16, NOL12, RBM12, ABT1, KRR1, HTATSF1, KIAA0020, NOP16, RPL27, UTP23, BST2, RBM47, EIF2C1, MAK16, PTCD1, DDX60, GPATCH4, PA2G4, DDX55, LARP7, GTF2E2, USP32, THUMPD1, MRPL30, DDX59, MRPS24, UCHL5, NXF3, AIMP1, THOC4, GLTSCR2, DHX33, KHDRBS2, BMS1, DDX1, NOC2L, MAP4, SAMD4A, YARS, RBM34, HDGF, PIWIL1, TRMU, ZFP36L1, TROVE2, ACTN4, RBM19, AK8, XRCC6, FAM208A, SRSF10, NAT10, SNRNP35, RPL7, MRPL3, NOP9, ARL6IP4, DDX52, HNRNPD, MRPS23, RPL15, HSP90AB1, RBM4, DHX30, HNRNPA1, NXF2, THRAP3, ADAD2, RPL13A, RPL35, HMGB2, HIST1H1C, EIF2C3, HNRNPC, ILF3, RBM15B, RPL19, MRPL4, DUS2L, TRIP6, GTPBP4, EEF2, DDX54, RPL23A, RRP36, PPAN, PATL1, REX04, DND1, NOP56, RPS11, RBM15, RPS4X, MRPS15, HNRNPR, PIWIL2, SYNCRIP, SNRPB, RPL32, or FBL.

25. (canceled)

26. The method of claim 1, wherein the RBP binds downstream of a poly(A) signal and/or site of the target RNA, and wherein the RBP activates alternative polyadenylation of the target RNA, optionally wherein the RBP is CSTF1, TOB2, PRRC2B, PARN, RBM11, DDX17, GTSF1, DDX5, RBM10, SNRPA, RBM22, CNOT4, RBM14, MBNL1, SUPT5H, ZC3HAV1, RBM4, CNOT2, SAMD4A, CDC40, HNRNPH1, SRP68, EIF4B, CSTF2T, LGALS3, NANOS3, SFPQ, CIR1, PPP1CA, ZMAT3, PRPF4, PRPF4, SCAF8, TRNAU1AP, EDC3, PCBP3, LSM1, PPIA, TPD52L2, RBM5, APOBEC3A, LGALS1, CDC42EP4, DZIP3, HNRNPH2, STAU2, PCBP1, GRB2, NUP35, EIF4H, BTG1, WDR6, THUMPD1, RSRC1, SMNDC1, SYMPK, NONO, MBNL2, MAZ, STAU1, HNRNPF, TFIP11, SMN1, AHNAK, YTHDF1, NGRN, METTL16, SNRPG, SF3A2, STAU2, YWHAE, YWHAG, SFRS2IP, PEG10, HRSP12, EXOSC10, CDC42EP4, LSM7, ZC3H7A, SNRPN, RBM42, AHNAK, GSPT1, SMN1, MECP2, SRSF9, QARS, ASCC3, CUGBP1, ZFC3H1, NMD3, SNRPB, SRSF2, NUSAP1, RBM38, CORO1A, SFRS17A, BTG4, GTPBP10, SNW1, or CPSF6.

27. (canceled)

28. The method of claim 1, wherein the RBP binds downstream of a poly(A) signal and/or site of the target RNA, and wherein the RBP inhibits poly(A) site selection of the target RNA, optionally wherein the RBP is FZD3, PPIG, CSTF3, WDR36, DHX8, SLIRP, CNOT10, SOX21, MRPS11, PURG, ADK, TRAP1, LSM4, NGDN, DYNC1H1, FLYWCH2, NPM1, IGF2BP1, ASS1, GNL2, RBPMS, LSM10, FAM46A, TPT1, RNMTL1, LARP4, CPSF5, LUC7L3, NAA15, RBM3, TPT1, ELAC2, RPGR, PNO1, UTP3, SNRPB2, HIST1H1C, ASCC1. SART3, EIF2C3, EIF4A1, EIF3L, MRPS15, LUC7L, SNRPE, LIN28A, CNOT10, NHP2, PARP12, HADHB, MRPS30, XPO5, RPS15A, NPM1, PURB, MTPAP, MRPL11, COA6, GLE1, NUDT16, GRN, RBM17, DHX57, PHF5A, SMG6, SARNP, SUGP2, TSR1, KRR1, C1D, ESRP1, PSMD4, SRSF10, TRMT10C, ZGPAT, BOLL, SPATS2L, MRPS24, RBM26, RPL6, CLASRP, DDX59, SEC63, EIF2S2, RPL23, DHX37, CPEB1, SNRPF, MRPS31, PNLDC1, MRPS23, RPS3A, TEFM, CCT4, POLR2G, RPS7, CNBP, FUBP1, SRPR, DDX60, NAP1L3, RPL22, EIF3C, DDX27, NOA1, LARP1B, FDPS, USP36, CMSS1, CCNL2, RPP30, SF3B3, MEPCE, SBDS, RPUSD4, U2AF2, RPL21, THOC3, FXR2, RPL6, LSM6, FAM98A, WDR33, DUS2L, THOC5, TRIM39, FAM208A, RRP7A, MRPS35, P4HB, LARP1, MAK16, RBM4, EIF2C2, ARL6IP4, FZD8, FTSJ3, PUS7, IFIT2, EIF2C2, S100A4, DHX58, TRIM56, SRPR, SERBP1, MRPL30, GTF2F1, LSMD1, TFB1M, FKBP3, PHF6, METTL3, ALDH18A1, ZFC3H1, GSPT2, GTPBP4, EXOSC3, PRPF6, RC3H2, NSUN2, DHX35, DCN, ANXA2, DHX40, SAFB2, RPS6, IFIH1, DDX43, NOL 12, GFM1, RPS20, PUF60, PIWIL1, RBM8A, LARP6, HSP90AB1, MRPS5, MAP4, MRPL37, PRPF19, XRN2, HFM1, ALDH6A1, FTO, SPATS2, TRMU, USP32, TROVE2, RPS15A, XRCC6, DNAJC17, NOP58, RPL7, RPL10A, DDX1, C16orf88, APOBEC3B, PPAPDC2, HDGF, C1orf131, SUPV3L1, RPS3, TDRD9, RALYL, CPSF2, SUCLG1, DDX20, RPN1, RPP25, PTRH1, EEF1A1, FAM32A, SEC63, CNOT6, ILF3, SRFBP1, TRIP6, NOSIP, FSCN1, NOB1, RPS6, GNL2, SRPK2, EIF3D, RPL15, TRMT6, RPL8, NOP2, CHD2, CIRBP, RBMS1, SERBP1, NOP16, FASTKD2, CPSF4, DDX11, CHTOP, PWP2, SREK1, LSM3, BCCIP, BYSL, SECISBP2, TRMT2A, REPIN1, CIRH1A, MRTO4, RPL19, HADHB, NOL10, SYF2, MFAP1, APOBEC3C, PRPF38B, RPS2, NSA2, CPSF3L, SNRNP35, THOC6, DDX11, RPL13A, DDX31, NPM1, BST2, PRPF40A, PES1, UCHL5, MKI67IP, FIP1L1, RPL8, MRPL42, ATP5C1, KRR1, RPL15, RPLP0, PUF60, ZRANB2, AIMP1, DDX54, MRPL4, SF3A1, POLR3E, RBM33, THRAP3, GNL3L, RPL4, DDX55, DHX33, RPL3, RPS8, DDX27, ERAL1, RRS1, PRR3, RNPC3, DDX50, RPL32, DDX39A, DDX39A, RPL5, RBFOX2, PPIG, RPS11, THOC4, RRP36, RPS4X, CCNL1, GLTSCR2, RBM46, RBM15, DHX16, CPSF4L, CPSF4, BMS1, BUD13, PRPF3, MRPL39, DKC1, KIAA0020, CLK3, RTN4, RPL23A, DDX18, RPL14, EDF1, CELF4, DDX55, SAMSN1, ZRSR2, DIEXF, RPL15, DHX30, SRSF4, SRP14, ZCRB1, LARP7, CPSF7, ELAVL2, FRG1, RPS24, DAZ3, NAT10, SRSF7, MKI67IP, DND1, DDX47, PA2G4, SRSF8, RPS4X, MRPL3, RSL1D1, PABPC5, NOC2L, APOBEC3D, UTP11L, G3BP2, ACAA2, DDX28, YTHDC1, RBM19, NOP9, SNIP1, RBM28, C1orf35, SFRS10, SFRS17A, CCDC59, RPL27, EIF2C1, NOL7, RBM34, LYAR, RPL28, KRR1, RBMX, PUF60, REXO4, PPAN, CCDC137, ZRSR1, SRSF12, ZRSR2, U2AF1, SRSF5, ADAD2, RBMX2, SRSF10, UTP23, SRSF6, ZC3H18, GPATCH4, HNRNPD, ABT1, TOP3B, HNRNPA1, RPL35, ELAVL4, RBM47, RRP8, RBM15B, PPIL4, ANXA2, RTCA, NOP56, GTF2E2, CPEB4, ZCCHC17, HNRNPR, PIWIL2, EEF2, RNPS1, RDBP, DDX49, RPL35, DDX52, FBL, ZFP36L1, DDX23, NOLC1, RBMX2, HNRNPCL1, TRA2A, HNRNPC, or HERC5.

29. (canceled)

30. An RNA regulation unit comprising an RNA binding protein (RBP) and a gene-targeting agent, wherein the RNA binding protein binds proximal to a poly(A) signal and/or site of a target RNA, optionally wherein the RNA regulation unit regulates alternative polyadenylation of RNA, and further optionally wherein the RNA binding protein (RBP) binds to the poly(A) sites, optionally wherein the RBP is not a CRISPR component.

31-40. (canceled)

41. The RNA regulation unit of claim 30, further comprising a delivery vehicle, optionally wherein the delivery vehicle comprises lipofection-based delivery or a virus-based delivery, and further optionally wherein the virus-based delivery comprises adeno-associated virus or lentivirus.

42-53. (canceled)

54. The method of claim 1, wherein the RBP is not a CRISPR component.

55. The method of claim 1, wherein the RBP activates poly(A) site selection of the target RNA and wherein the RBP is selected from CSTF1, TOB2, PRRC2B, PARN, RBM11, DDX17, GTSF1, DDX5, RBM10, SNRPA, CPSF5, RNPS1, CPSF6, CSTF1, RBM11, TRNAU1AP, RBM14, MBNL1, or PRRC2B.
